(19) United States
(12) Patent Application Publication
(10) Pub. No.: US 2024/0182922 A1
(43) Pub. Date: Jun. 6, 2024

(54) VIRAL VECTORS WITH REDUCED IMMUNOGENICITY

(71) Applicants: CORNELL UNIVERSITY, Ithaca, NY (US); UNIVERSITY OF WASHINGTON, Seattle, WA (US)

(72) Inventors: Shaoyi JIANG, Ithaca, NY (US); Zhefan YUAN, Ithaca, NY (US); Sijin LUOZHONG, Ithaca, NY (US); Wenchao GU, Ithaca, NY (US); Ruoxin LI, Ithaca, NY (US); Bowen LI, Seattle, WA (US)

(21) Appl. No.: 18/283,929

(22) PCT Filed: Mar. 25, 2022
(86) PCT No.: PCT/US22/21929

§ 371 Date: Sep. 25, 2023

Related U.S. Application Data
(60) Provisional application No. 63/166,417, filed on Mar. 26, 2021.

(51) Int. Cl. C12N 15/86; A61K 47/69; A61K 48/00

(52) U.S. Cl. CPC C12N 15/86; A61K 47/6901; A61K 48/0041; A61K 48/0083; C12N 2750/14143; C12N 2750/14145

(57) ABSTRACT

Compositions and methods for modified viral vectors which mitigate the immunogenicity of viral vectors, enabling multiple administrations of viral-based gene delivery viral vectors are described herein. The viral vectors advantageously possess low immunogenicity and comprise at least one immunosuppressive moiety. Also described herein are methods for introducing genetic material into a cell. Additionally, described herein are methods for preparing a modified viral vector. Lastly, described herein are methods for treating a subject, the method comprising administering to a subject a modified viral vector composition as described above.

### CROSS REFERENCE TO RELATED APPLICATION

This application claims the benefit of U.S. Provisional Patent Application No. 63/166,417, filed Mar. 26, 2021, the contents of which is incorporated herein by reference in its entirety.

### STATEMENT OF GOVERNMENT FUNDING

The subject of this disclosure was made with government support under Grant No. DMR-2002940 awarded by the National Science Foundation. The government has certain rights in the invention.

### BACKGROUND

Gene therapy mediated by virus vectors is one of the most promising approaches for the treatment of a variety of inherited and acquired diseases. Single AAV administration usually lasts from months to several years of gene expression above therapeutic levels. See, e.g., Verdera, H. C.; Kuranda, K.; Mingozzi, F., AAV Vector Immunogenicity in Humans: A Long Journey to Successful Gene Transfer. Mol. Ther. 2020, 28 (3), 723-746. Yet, many inherited diseases require lifelong treatment to avoid irreversible tissue damages (Jackson, M.; et al., The genetic basis of disease. Essays Biochem. 2018, 62 (5), 643-723). Increasing administration dosage may extend the therapeutic windows, but concern about potential toxicities rises (Khabou, H.; et al., Thresholds and Influence of Transgene Cassette in Adeno-Associated Virus-Related Toxicity. Hum. Gene Ther. 2018, 29 (11), 1235-1241; and Hinderer, C.; et al., Severe Toxicity in Nonhuman Primates and Piglets Following High-Dose Intravenous Administration of an Adeno-Associated Virus Vector Expressing Human SMN. Hum. Gene Ther. 2018, 29 (3), 285-298). Thus, the ability to re-administer AAV is crucial to achieve sustained therapeutic efficacy over time. However, vector immunogenicity represents a major limitation to re-administration of viral vectors. Persistent high-titer antibodies are triggered by multiple vector administrations, which abolishes any benefit of repeated viral vector-based treatments.

In some cases, a subject can exhibit an unwanted immunogenic response when a conventional viral vector is introduced into a subject. Although AAVs are considered low immunogenic and safe as compared with other viral vectors, the immunogenicity of capsids still represents a major obstacle to the re-administration of AAV vectors (Verdera, H. C.; et al., AAV Vector Immunogenicity in Humans: A Long Journey to Successful Gene Transfer. Mol. Ther. 2020, 28 (3), 723-746). Both humoral and cell-mediated immunities are observed in preclinical animal studies and human patients (Boutin, S.; et al., Prevalence of Serum IgG and Neutralizing Factors Against Adeno-Associated Virus (AAV) Types 1, 2, 5, 6, 8, and 9 in the Healthy Population: Implications for Gene Therapy Using AAV Vectors. Hum. Gene Ther. 2010, 21 (6), 704-712; and Calcedo, R.; Wilson, J. M., Humoral Immune Response to AAV. Front. Immunol. 2013, 4, 341). Disclosed herein are methods and compositions to reduce this unwanted immunogenic response to viral vector-mediated treatment. Compositions and methods are disclosed that focus on mitigating the immunogenicity of viral vector and enabling multiple administrations of viral-based gene delivery.

### SUMMARY

The present disclosure is foremost directed to compositions and methods which mitigate the immunogenicity of viral vectors, enabling multiple administrations of viral vectors such as gene delivery viral vectors. The viral vectors described herein advantageously possess low immunogenicity and comprise at least one immunosuppressive moiety (ISM).

In a first aspect, the present disclosure is directed to a modified viral vector, containing at least a viral vector (VV); and at least an immunosuppressive moiety (ISM) covalently linked directly or through a linker to the viral vector.

In some embodiments, the viral vector is a virus selected from the group consisting of retroviruses, lentiviruses, adenoviruses, and adeno-associated viruses (AAV). In some embodiments, the viral vector is an AAV selected from the group consisting of AAV1, AAV2, AAV3, AAV4, AAV5, AAV6, AAV7, AAV8, AAV9, AAV10, AAV11, AAV6.2, AAVrh10, AAV-DJ, AAV-DJ/8, AAV-PHP.B, AAV-PHP.eB, AAV-PHP.S, AAV2-retro, AAV2-QuadYF, AAV2.7m8, and genetically engineered derivatives thereof.

In some embodiments, the immunosuppressive moiety (ISM) comprises one or more compounds selected from the group consisting of small molecules, polymeric molecules, and peptides, wherein the small molecules, polymeric molecules, and peptides have a molecular weight of 100-10,000 g/mol.

In some embodiments, the ISM comprises a phosphoserine (PS) having the following structure:

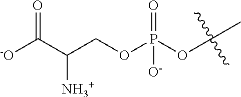

wherein the wavy line indicates a bond to a linker or a direct bond to the viral vector.

In some embodiments, the ISM comprises polysialic acid (PSA). In some embodiments, the PSA comprises the following structure:

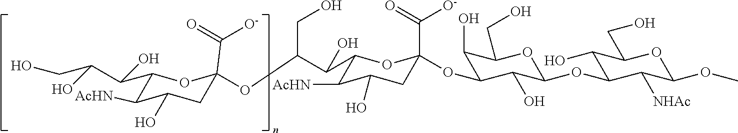

wherein Ac represents acetyl; and n is at least 2.

In some embodiments, the ISM one or more mTOR inhibitors such as Rapamycin, Temsirolimus, Everolimus, Umirolimus, and combinations thereof.

In some embodiments, the ISM comprises one or more selected from the group consisting of, aryl hydrocarbon receptor (AHR) ligands, vitamin D3, retinoic acid, peptides with CxxC/CxxS flanking epitope where x is any amino acid, and combinations thereof.

In some embodiments, the ISM comprises one or more molecules from apoptotic cells such as phosphatidylserine, chromatin oligonucleotide, and combinations thereof.

In some embodiments, the ISM comprises one or more secondary lymphoid organs (spleen or lymph nodes) or liver targeting moieties such as N-acetylgalactosamine (GalNAc), N-Acetylglucosamine (GlcNAc), N-acetylneuraminic acid (NeuAc or sialic acid), galactose, and fucose, and combinations thereof.

In some embodiments, the ISM comprises one or more inflammation reducing moieties such as Z2-Y12, Z1-Y15, Z1-Y19, dexamethasone, lymphocyte function-associated antigen antagonist, d-mannose, and combinations thereof.

In some embodiments, the viral vector and immunosuppressive moiety are covalently linked to each other directly.

In some embodiments, the viral vector and immunosuppressive moiety are covalently linked through a linker. In a fourteenth set of embodiments, the linker comprises a linker peptide and/or a crosslinker compound.

In some embodiments, the linker peptide is a peptide of 25 amino acids or less. In some embodiments, the linker peptide comprises alternating Glu-Lys (EK) peptides or Lys-Lys (KK) peptides. In some embodiments, the linker peptide comprises (KK)8-C-NH2, or a derivative thereof.

In some embodiments, the crosslinker compound comprises a N-hydroxysuccinimide ester-Maleimide heterobifunctional aliphatic reagent such as AMAS, BMPS, GMBS, Sulfo-GMBS, MBS, Sulfo-MBS, SMCC, Sulfo-SMCC, EMCS, Sulfo-EMCS, SMPB, Sulfo-SMPB, SMPH, LC-SMCC, and Sulfo-KMUS.

In some embodiments, the modified viral vector comprises a plurality of the linker. In some embodiments, each linker comprises a plurality of a peptide linker and/or a plurality of a crosslinker compound.

In some embodiments, viral vector comprises surface sites to which the immunosuppressive moiety or the linker covalently binds such as capsid proteins, gag proteins, envelop proteins, and/or lipid layers.

In some embodiments, the modified viral vector comprises the following structure:

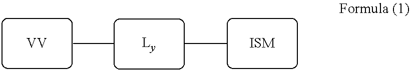

wherein:
VV is the viral vector;
L is a linear or branched linker selected from the group consisting of peptides, saccharides, lipids, and non-biological molecules and polymers, wherein y is 0 or 1, which corresponds to the absence or presence of the linker, respectively;
ISM is the immunosuppressive moiety; and
z is at least 1, wherein z corresponds to the number of ISM attached to L;
wherein the lines connecting VV, L_{y}, and ISM represent covalent bonds.

In some embodiments, the linker or ISM is attached to the viral vector via an amino group of the viral vector. In some embodiments, the amino group is on a capsid or envelope of the viral vector.

In some embodiments, the linker is present and the modified viral vector comprises the following structure:

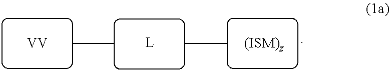

In some embodiments, the linker is attached to the viral vector via an amino group of the viral vector. In some embodiments, the amino group is on a capsid or envelope of the viral vector.

In some embodiments, the modified viral vector comprises the following structure:

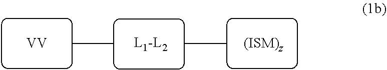

wherein:
VV is the viral vector;
L₁ and L₂ are portions of the linear or branched linker L, wherein L₁ represents a bifunctional crosslinker possessing an amino-reactive and thiol-reactive group, wherein the amino reactive group is bound to an amino group of the viral vector; and L₂ represents a linking portion containing a thiol group bound to the thiol-reactive group of L₁, wherein L₂ is also bound to the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

In some embodiments, the modified viral vector comprises the following structure:

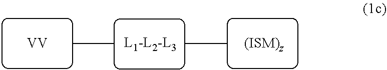

wherein:
VV is the viral vector;
L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is selected from the group consisting of peptides, saccharides, lipids, and non-biological molecules and polymers; and L₃ represents a bifunctional crosslinker possessing an amino-reactive and thiol-reactive group, wherein the amino reactive group is bound to an amino group of L₂ and the thiol-reactive group is bound to a thiol group of the ISM, or wherein the amino reactive group is bound to an amino group of the ISM and the thiol-reactive group is bound to a thiol group of L₂;
ISM is the immunosuppressive moiety; and
z is at least 1, wherein z corresponds to the number of ISM attached to L.

In some embodiments, the modified viral vector comprises the following structure:

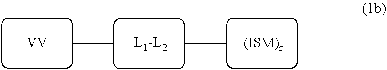

wherein:
VV is the viral vector;
L₁ and L₂ are portions of the linear or branched linker L, wherein VV is modified to contain a thiol group, and L₁ represents a thiol-reactive group bound to the thiol group of VV, wherein L₂ is bound to L₁ and the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

In some embodiments, the modified viral vector comprises the following structure:

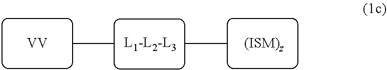

wherein:
VV is the viral vector;
L₁, L₂, and L₃ are portions of the linear or branched linker L, wherein VV is modified to contain a thiol group, and L₁ represents a thiol-reactive group bound to the thiol group of VV; L₃ represents a bifunctional crosslinker possessing an amino-reactive and thiol-reactive group, wherein the amino reactive group is bound to an amino group of L₂ and the thiol-reactive group is bound to a thiol group of the ISM, or the amino reactive group is bound to an amino group of the ISM and the thiol-reactive group is bound to a thiol group of L₂;
wherein L₂ is selected from the group consisting of peptides, saccharides, lipids, and non-biological molecules and polymers;
ISM is the immunosuppressive moiety; and
z is at least 1, wherein z corresponds to the number of ISM attached to L.

In some embodiments, the modified viral vector comprises the following structure:

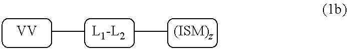

wherein:
VV is the viral vector;
L₁ and L₂ are portions of the linear or branched linker L, wherein VV and L₂ are modified to contain an azide or alkyne group in order for VV and L₂ to attach by azide-alkyne cycloaddition click chemistry;
L₁ represents a 1,2,3-triazole group connecting VV and L₂, wherein the 1,2,3-triazole group is the result of an azide-alkyne cycloaddition click chemistry reaction between the azide or alkyne group on the VV and alkyne or azide group, respectively, on L₂; wherein L₂ is bound to L₁ and the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

In some embodiments, the modified viral vector comprises the following structure:

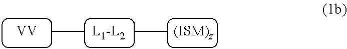

wherein:
VV is the viral vector;
L₁ and L₂ are portions of the linear or branched linker L, wherein L₁ and ISM are modified to contain an azide or alkyne group in order for L₁ and ISM to attach by azide-alkyne cycloaddition click chemistry;
L₁ is selected from the group consisting of peptides, saccharides, lipids, and non-biological molecules and polymers; L₂ represents a 1,2,3-triazole group connecting L₁ and ISM, wherein the 1,2,3-triazole group is the result of an azide-alkyne cycloaddition click chemistry reaction between the azide or alkyne group on L₁ and alkyne or azide group, respectively, on ISM;
ISM is the immunosuppressive moiety; and
z is at least 1, wherein z corresponds to the number of ISM attached to L.

In some embodiments, the linker comprises a peptide.

In some embodiments, the peptide comprises polylysine. In some embodiments, the peptide contains no more than 25 amino acid units.

In some embodiments, the modified viral vector comprises more than one immunosuppressive moiety covalently bound to the viral vector. In some embodiments, the modified viral vector comprises 1-10,000 immunosuppressive moieties covalently bound to the viral vector. In some embodiments, the modified viral vector comprises 1-5,000 immunosuppressive moieties covalently bound to the viral vector. In some embodiments, the modified viral vector comprises 1-2,000 immunosuppressive moieties covalently bound to the viral vector. In some embodiments, the modified viral vector comprises 100-2,000 immunosuppressive moieties covalently bound to the viral vector. In some embodiments, the modified viral vector achieves a transfection efficiency that is at least 30% of the transfection efficiency by an unmodified viral vector. In some embodiments, the modified viral vector achieves a transfection efficiency that is at least 40% of the transfection efficiency by an unmodified viral vector. In some embodiments, the modified viral vector achieves a transfection efficiency that is at least 50% of the transfection efficiency by an unmodified viral vector. In some embodiments, the modified viral vector achieves a transfection efficiency that is at least 60% of the transfection efficiency by an unmodified viral vector. In some embodiments, the modified viral vector achieves a transfection efficiency that is at least 70% of the transfection efficiency by an unmodified viral vector.

In some embodiments, for any of the formulas provided above, the ISM is or includes phosphoserine (PS).

In particular embodiments, the modified viral vector has the following structure:

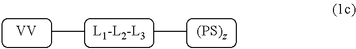

wherein: VV is the viral vector; phosphoserine (PS) is modified to contain a thiol group, and multiple PS moieties are present; z is greater than 1 and corresponds to the number of PS moieties attached to L₂ via L₃; L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is a polypeptide containing multiple amino groups; and L₃ represents a multiplicity of bifunctional crosslinkers each possessing an amino-reactive and thiol-reactive group, wherein the amino reactive group is bound to the amino groups of L₂ and the thiol-reactive group is bound to the thiol groups of the multiple PS moieties.

In other particular embodiments, the modified viral vector has the following structure:

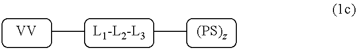

wherein: VV is the viral vector; phosphoserine (PS) is modified to contain a thiol group; L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is a polypeptide; L₂ and PS are modified to contain an azide or alkyne group in order for L₂ and PS to attach by azide-alkyne cycloaddition click chemistry and L₃ represents a 1,2,3-triazole group connecting L₂ and PS, wherein the 1,2,3-triazole group is the result of an azide-alkyne cycloaddition click chemistry reaction between the azide or alkyne group on L₂ and alkyne or azide group, respectively, on PS; and z is at least 1, wherein z corresponds to the number of PS attached to L₂.

In some embodiments, for any of the formulas provided above, the ISM is or includes polysialic acid (PSA).

In particular embodiments, the modified viral vector has the following structure:

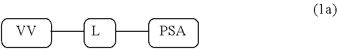

wherein: VV is the viral vector, modified to contain a thiol group; PSA is polysialic acid; L is a linker connecting VV and PSA and comprises a thiol-reactive group bound to the thiol group of VV.

In other particular embodiments, the modified viral vector has the following structure:

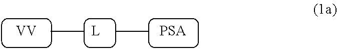

wherein: VV is the viral vector, modified to contain an alkyne or azide group; PSA is polysialic acid, modified to contain an alkyne or azide group; L is a 1,2,3-triazole group connecting VV and PSA, wherein the 1,2,3-triazole group is the result of an azide-alkyne cycloaddition click chemistry reaction between the azide or alkyne group on VV and alkyne or azide group, respectively, on PSA.

In another aspect, the present disclosure is directed to a method for preparing a modified viral vector, the method comprising attaching an immunosuppressive moiety to a viral vector to obtain a modified viral vector described herein.

In still another aspect, the present disclosure is directed to a method for introducing genetic material into a cell, the method comprising contacting a cell with a modified viral vector described herein. In some embodiments, the method comprises contacting the cell multiple times with a modified viral vector described herein. In some embodiments, the method comprises contacting the cell multiple times with more than one modified viral vector described herein.

In another aspect, the present disclosure is directed to a method for treating a subject, the method comprising administering to a subject a modified viral vector described herein. In some embodiments, the subject exhibits a reduced immune response after the subject is administered a modified viral vector described herein as compared to a control subject administered an unmodified viral vector. In some embodiments, the method comprises administering a single modified viral vector described herein to a subject multiple times. In some embodiments, the method comprises administering more than one modified viral vectors described herein. In some embodiments, the subject exhibits a reduced immune response as compared to a control subject administered an unmodified viral vector. In some embodiments, the subject is administered with (i) a modified viral vector at a first point in time and subsequently (ii) the modified viral vector at a second point in time, and the subject exhibits a reduced immune response as compared to a control subject administered an unmodified viral vector at the first and second points in time. In some embodiments, the subject is administered with (i) a modified viral vector at a first point in time, and subsequently (ii) a different modified viral vector at a second point in time; and the subject exhibits a reduced immune response as compared to a control subject administered an unmodified viral vector at the first and second points in time. In some embodiments, the second point in time is between 1 day and 49 days after the first point in time. In some embodiments, the second point in time is at least 21 days after the first point in time.

### BRIEF DESCRIPTION OF THE DRAWINGS

FIG. 1A is an illustration of the conjugation strategy for preparing PS containing peptide conjugated AAV vectors.

FIG. 1B is a top view of the structure of VP3 protein from AAV serotyped 8 (PDB #: 2qa0).

FIG. 2A is a graph representing percent transfection of AAV8 and KKPS-AAV8 vectors in vitro studies.

FIG. 2B shows GFP expression in mouse liver sections that were taken after 3 weeks of single dose injection of AAV8-CAG-GFP.

FIG. 2C shows GFP expression in mouse liver sections that were taken after 3 weeks of single dose injection of KKPS-AAV8-CAG-GFP;

FIG. 3A shows a whole brain eGFP expression delivered by KKPS-AAV php.eb-CAG-eGFP vectors.

FIG. 3B shows cells and neurons in cortex region of the brain expressing eGFP delivered by KKPS-AAV php.eb-CAG-eGFP vectors.

FIG. 3C shows cells and neurons in hippocampus region of the brain expressing eGFP delivered by KKPS-AAV php.eb-CAG-eGFP vectors.

FIG. 3D shows cells and neurons in thalamus region of the brain expressing eGFP delivered by KKPS-AAV php.eb-CAG-eGFP vectors.

FIG. 3E and FIG. 3F show a comparison of representations of whole brain eGFP expression delivered by AAV php.eb-CAG-eGFP vectors (FIG. 3E) and by KKPS-AAV php.eb-CAG-eGFP vectors (FIG. 3F).

FIG. 4A shows IVIS images of luciferase expression in C57bl/6 mice using unmodified AAV8 and KKPS-AAV8, respectively. Mice were IV injected with single dose of native or modified AAV8-CMV-Fluc (4*1012 vg/kg) on Day 1. On Day 21 and 28, mice were i.p. injected with D-luciferin (150 mg/kg) and imaged in an IVIS system (PerkinElmer).

FIG. 4B is a graph summarizing the data of total luminescence as seen in FIG. 4A.

FIG. 5A shows administration route of two-dose cohort for immunogenicity study.

FIG. 5B is a graph representation of flow cytometry analysis of anti-AAV8 IgG titers, where conjugation of KKPS successfully mitigated the generation of anti-AAV8 antibody (titers: 800) while native AAV8 showed highest antibody titers (>6400).

FIG. 5C is a summary of the percentage of Treg phenotype (Foxp3+) cells among CD4+CD25+ splenocytes.

FIG. 5D is a summary of the percentage of activated Gemina center B cells.

FIG. 5E AAV8-specific mouse Interferon gamma ELISPOT;

FIG. 5F shows anti-AAV8 IgG secreting B cell ELISPOT.

FIG. 6A shows rAAV construct encoding human B domain depleted FVIII.

FIG. 6B shows FVIII gene delivery in Hemophilia A mice (FVIII knockout). A model AAV8 vector encoding luciferase (4*1012 vg/kg) were i.v. injected into mice on Day 1. Mice were received with 2^{nd} AAV8 vector encoding hFVIII (4*1012 vg/kg). Plasma was collected on Day 49 and 56. Tail bleeding test was also performed on Day 56. After that, all the mice were sacrificed immediately.

FIG. 6C is a graph representing FVIII activities in plasma, where the data was normalized with standard FVIII activity tested from pooled health human plasma.

FIG. 7 is a schematic showing the preparation of PSA-NH2 and modified AAV.

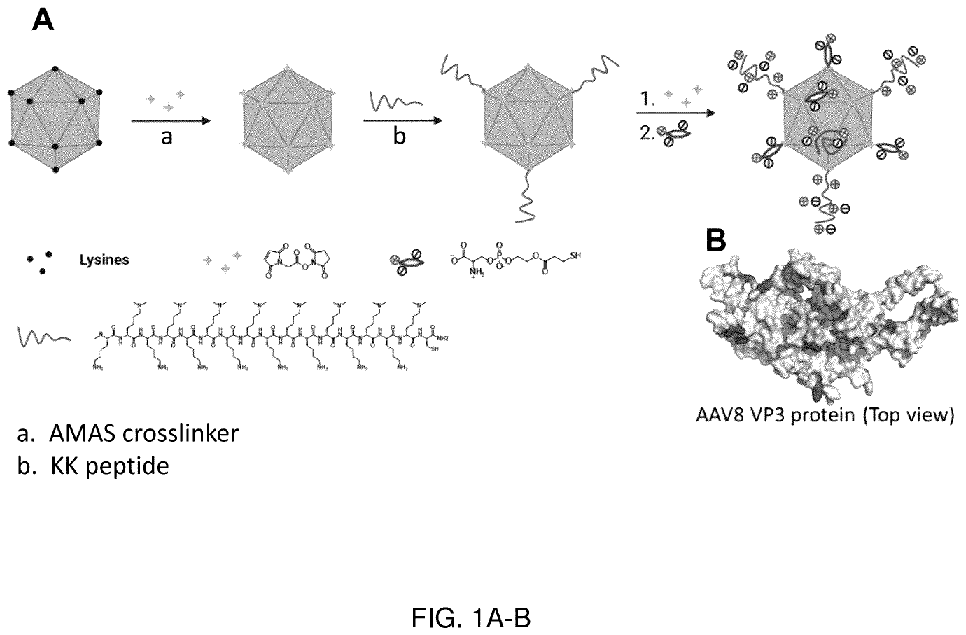

### DETAILED DESCRIPTION

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

In some cases, a subject can exhibit an unwanted immunogenic response when a conventional viral vector is introduced into a subject. This application discloses methods and compositions to reduce this unwanted immunogenic response. In some embodiments, this application discloses modified viral vectors that comprise a viral vector with a covalently linked immunosuppressive moiety (ISM). In some embodiments, the viral vector is directly linked to the immunosuppressive moiety. In some embodiments, the viral vector is linked to the immunosuppressive moiety through a linker. In some embodiments, the modified viral vectors can reduce and/or prevent unwanted immune responses in a subject when the modified viral vector is administered to the subject as compared to an unmodified viral vector (i.e., viral vectors without an immunosuppressive moiety). Methods disclosed herein also include preparing the modified viral vectors. Methods disclosed herein also include administering a modified viral vector to a subject.

Viruses comprise a viral genome, a capsid, and sometimes an outer envelope surrounding the capsid. The capsid comprises capsomeres, protein subunits which include hexons, penton base proteins, and fibers. The envelope comprises protein and phospholipid membranes. Both the capsid and envelope assist the virus in attaching to host cells through their surface components, such as glycoproteins and matrix proteins.

### Viral Vectors

As used herein, “a viral vector” refers to a virus-based or virus-derived composition that has the ability to act as a carrier of a heterologous molecule of interest, such as a heterologous nucleic acid. A heterologous nucleic acid can be inserted in the genomic nucleic acid of a virus which is introduced to a recipient. In some embodiments, the viral vector is a virus in which the viral genome has been manipulated to accommodate a nucleic acid sequence that is non-native with respect to the viral genome. Typically, a viral vector is generated by introducing one or more mutations (e.g., a deletion, insertion, or substitution) into the viral genome of the virus so as to accommodate the insertion of a non-native nucleic acid sequence, for example, for gene transfer, into the virus. In the context of the present disclosure, a viral vector includes a virus, or viral particle, which comprises a viral genome. The genome of the virus may be modified to contain a minimum of components for the assembly of a functional recombinant virus, or viral particle, which is loaded with or engineered to express or deliver a desired payload, which may be delivered to a target cell, tissue, organ, or organism.

In some embodiments, the viral genome comprises a heterologous polynucleotide, e.g., an RNA or DNA molecule, which acts as a therapeutic agent. In some embodiments, the heterologous polynucleotide encodes or otherwise produces a polynucleotide that is processed into small double stranded RNA (dsRNA) molecules (small interfering RNA, siRNA, miRNA, pre-miRNA) targeting a gene of interest. In some embodiments, the heterologous polynucleotide comprises a gene of interest, e.g., a gene known to be associated with a targeted disease, such as blood diseases and cancers, cystic fibrosis, muscular dystrophy, and several central nervous system (CNS) disorders including Parkinson's, Alzheimer's disease, Batten disease, Friedreich's Ataxia, and genetic amyotrophic lateral sclerosis (ALS). In some embodiments, the gene of interest is functionally classified as an information storage and processing gene. In some embodiments, the gene of interest is functionally classified as a cellular processes and signaling gene. In some embodiments, the gene of interest is functionally classified as a metabolism gene. In some embodiments, the gene of interest is known as a protein coding gene. In some embodiments, the gene of interest is aromatic 1-amino acid decarboxylase (AADC), neuronal ceroid lipofuscinoses (NCLs) including CLN2 and CLN6, N-acetyl-alpha-glucosaminidase (NAGLU), Glial Cell Derived Neurotrophic Factor (GDNF), Neurturin (NRTN), survival motor neuron (SMN), Gigaxonin (GAN), Cyclic Nucleotide Gated Channel Subunit Beta 3 (CNGB3), replication (REP) gene of the human parvovirus adeno-associated virus (AAV), CHM Rab Escort Protein (CHM), Retinoid Isomerohydrolase RPE65 (RPE65), NADH dehydrogenase subunit 4 (ND4), Retinaldehyde Binding Protein 1 (RLBP1), Retinitis Pigmentosa GTPase Regulator (RPGR), Retinoschisin 1 (RS1), UDP Glucuronosyltransferase Family 1 Member A1 (UGT1A1), low density lipoprotein receptor (LDLR), glucose-6-phosphatase catalytic-subunit-encoding genes (G6PC, G6PC2, and G6PC3), FVIII gene, FIX gene, zinc-finger nuclease (ZFN1 and ZFN2), N-Sulfoglucosamine Sulfohydrolase (SGSH), Arylsulfatase B (ARSB), SERPINA1 gene, Neurotrophin 3 (NTF3), Myotubularin 1 (MTM1), and acid alpha-glucosidase (GAA). In some embodiments, the heterologous polynucleotide comprises DNA that transcribes Cas nuclease mRNA and/or guide RNA nucleic acid. The guide RNA nucleic acid may be, for example, a single-guide RNA (sgRNA). In some embodiments, the heterologous polynucleotide comprises DNA templates that transcribe single-stranded, 5′-capped messenger RNA (mRNA), encoding the viral spike (S) protein of SARS-CoV-2.

In some embodiments, the viral vector is a virus selected from retroviruses, lentiviruses, adenoviruses (Ad), adeno-associated viruses (AAV), or their genetic engineered derivatives.

By “genetic engineered derivatives” of a virus, it is meant viruses that are generated through genetic modification, which involves the directed insertion, deletion, artificial synthesis, or change of nucleotide sequences in viral genomes using biotechnological methods known to those of skill in the art. The “genetic engineered derivatives” of a virus refers to a modified virus (in relation to a native or starting virus) or a molecule or moiety resembling a native or starting virus in structure and/or function. Virus variants or derivatives may be altered in their amino acid sequence, composition, or structure as compared to a native or starting virus.

As used herein, the term “variants” or “variant” refers to a nucleic acid or polypeptide differing from a reference nucleic acid or polypeptide yet retaining essential properties thereof. Generally, variants are overall closely similar, and, in many regions, identical to the reference nucleic acid or polypeptide.

In some embodiments, the viral vector is a retrovirus or a genetic engineered derivative thereof. Retroviruses are double stranded RNA enveloped viruses mainly characterized by the ability to “reverse-transcribe” their genome from RNA to DNA. Retroviruses contain a dimeric genome of identical positive RNA strands complexed with the nucleocapsid proteins. The genome is enclosed in a protein capsid that also contains enzymatic proteins, namely the reverse transcriptase, the integrase and proteases, required for viral infection. The matrix proteins form a layer outside the capsid core that interacts with the envelope, a lipid bilayer derived from the host cellular membrane, which surrounds the viral core particle. Anchored on this bilayer, are the viral envelope glycoproteins responsible for recognizing specific receptors on the host cell and initiating the infection process. Envelope proteins are formed by two subunits, the transmembrane (TM) that anchors the protein into the lipid membrane and the surface (SU) which binds to the cellular receptors. Retroviruses encode four genes: gag (group specific antigen), pro (protease), pol (polymerase) and env (envelope). The gag sequence encodes the three main structural proteins: the matrix protein, nucleocapsid proteins, and capsid protein. The pro sequence encodes proteases responsible for cleaving Gag and Gag-Pol during particle assembly, budding and maturation. The pol sequence encodes the enzymes reverse transcriptase and integrase, the former catalyzing the reverse transcription of the viral genome from RNA to DNA during the infection process and the latter responsible for integrating the proviral DNA into the host cell genome. The env sequence encodes for both SU and TM subunits of the envelope glycoprotein.

In some embodiments, the viral vector is a lentivirus or a genetic engineered derivative thereof. Lentiviruses are complex retroviruses which, in addition to the common retroviral genes gag, pol and env, contain other genes with regulatory or structural function. Lentiviruses have the ability to integrate into non-dividing cells. The lentiviral genome and the proviral DNA have the three genes found in retroviruses; gag, pol and env, which are flanked by two LTR sequences. The gag gene encodes the internal structural (matrix, capsid and nucleocapsid) proteins; the pot gene encodes the RNA-directed DNA polymerase (reverse transcriptase), a protease and an integrase; and the env gene encodes viral envelope glycoproteins.

In some embodiments, the viral vector is an adenovirus (or “Ad”). Adenovirus is a medium-sized (90-100 nm), nonenveloped icosohedral virus containing approximately 36 kb of double-stranded DNA. The adenovirus capsid mediates the key interactions of the early stages of the infection of a cell by the virus. The adenovirus capsid is required for packaging adenovirus genomes at the end of the adenovirus life cycle. The capsid comprises capsomeres, which include hexons, penton base proteins, and fibers. The hexon comprises three identical proteins, namely polypeptide II. The penton base comprises five identical proteins and the fiber comprises three identical proteins. Proteins IIIa, VI, and IX are present in the adenoviral coat and are believed to stabilize the viral capsid. The expression of the capsid proteins, with the exception of pIX, is dependent on the adenovirus polymerase protein. Therefore, major components of an adenovirus particle are expressed from the genome only when the polymerase protein gene is present and expressed.

In some embodiments, the viral vector is an adeno-associated viruses (AAV)virus or a genetic engineered derivative thereof. AAV vectors may include the viral genome, in whole or in part, of any naturally occurring and/or recombinant AAV serotype nucleotide sequence or variant. Serotypes of AAV include, but are not limited to AAV1, AAV2, AAV3, AAV4, AAV5, AAV6, AAV7, AAV8, AAV9, AAV10, AAV11, AAV6.2, AAVrh10, AAV-DJ, AAV-DJ/8, AAV-PHP.B, AAV-PHP.eB, AAV-PHP.S, AAV2-retro, AAV2-QuadYF, AAV2.7m8 and their genetic engineered derivatives. AAV variants may have sequences of significant homology at the nucleic acid (genome or capsid) and amino acid levels (capsids), to produce constructs which are generally physical and functional equivalents, replicate by similar mechanisms, and assemble by similar mechanisms. In some embodiments, the AAV is an AAV empty capsid. In some embodiments, the AAV is a single-stranded AAV. In some embodiments, the AAV is a self-complementary AAV. In some embodiments, the AAV comprise AAV that infect humans. In some embodiments, the AAV comprise AAV that infect a non-human primate. In some embodiments, the AAV comprise AAV that infect mammals. In some embodiments, serotypes of Ad include but not limited to Human adenovirus A, B, C, D, E, and F. In some embodiments, serotypes of Ad include but are not limited to Adenoviruses 1-51. Many strains of AAV have been identified in nature. They are divided into different serotypes based on different antigenicity of the capsid protein on the viral surface. Different serotypes can render the virus with different tissue tropism (i.e., tissue specificity of infection).

In some embodiments, the viral vector comprises nucleic acids, such as the genomic nucleic acids of a virus, optionally with a heterologous polynucleotide. In some embodiments, the nucleic acids comprise DNA, RNA, and/or hybrids thereof. In some embodiments, the nucleic acids comprise polynucleotides in either single-stranded or double-stranded form. In some embodiments, the polynucleotides comprise plasmid DNA or linearized DNA. In some embodiments, the polynucleotides comprise messenger RNA (mRNA), small interfering RNA (siRNA), microRNA (miRNA), circularRNA (circRNA), long-noncoding RNA (lncRNA), and antisense oligonucleotide (ASO). In some embodiments, the nucleic acid encodes fusion biological moieties comprising protective domains and functional domains. In some embodiments, functional domains are fused directly or via a linker consisting of amino acids to the protective domains. In some embodiments, the protective domain of the fusion biological moieties is a domain or domains comprising, a) a plurality of negatively charged amino acids (e.g., aspartic acid, glutamic acid, and derivatives thereof) and b) a plurality of positively charged amino acids (e.g., lysine, histidine, arginine, and derivatives thereof); and/or additional amino acids independently selected from the group consisting of proline, serine, threonine, asparagine, glutamine, glycine, and derivatives thereof. In some embodiments, the ratio of the number of negatively charged amino acids to the number of positively charged amino acids is from about 1:0.5 to about 1:2. In some embodiments, the protective domain of the fusion biological moieties comprises XTEN and/or proline-alanine-serine and elastin-like polypeptides. In some embodiments, the protective domain of the biological moieties comprises natural half-life extension domains like Fc fragment and albumin.

### Immunosuppressive Moiety

The term “immunosuppressive moiety,” as used herein, includes any molecule or moiety that has the ability to inhibit, suppress, or prevent one or more functions or activities of the immune system of a subject. In some embodiments, the immunosuppressive moiety suppresses the immune system by suppressing cellular immunity. As a result of the ability of a modified viral vector to suppress cellular immunity, any immune response induced in a recipient by the viral vector is reduced (reduced immunogenicity) as compared to an unmodified viral vector. In some embodiments, the immunosuppressive moiety inhibits the activation of T cells. In some embodiments, the immunosuppressive moiety upregulates regulatory T cells (Treg), for example, by enhancing the function of Treg, as reflected by, e.g., reduced induction and proliferation of effector T cells. In some embodiments, the immunosuppressive moiety reduces production of antibodies against a viral vector. In some embodiments, the immunosuppressive moiety reduces production of antibodies against AAVs. In some embodiments, the immunosuppressive moiety reduces inflammation in the subject. In some embodiments, the immunosuppressive moiety targets secondary lymphoid and liver. In some embodiments, the immunosuppressive moiety inhibits mammalian target of rapamycin (mTOR). In some embodiments, the immunosuppressive moiety comprises a combination of inhibition of T cell activation, upregulation of Treg, and/or reduction of antibodies to the viral vector. In some embodiments, the immunosuppressive moiety comprises a combination of cellular immunity suppressors, inflammation reducers, secondary lymphoid and liver targeting moieties and/or mTOR inhibitors.

In some embodiments, the immunosuppressive moiety suppresses the immune system by suppressing cellular immunity. In some embodiments, suppressing cellular immunity comprises inhibiting T cell activation. In some embodiments, suppressing cellular immunity comprises inhibiting cytokine release. In some embodiments, the immunosuppressive moiety suppressing cellular immunity comprises one or more of lymphocyte function-associated antigen antagonist, sialic acid, aryl hydrocarbon receptor (AHR) ligands, dexamethasone, vitamin D3, d-mannose, retinoic acid, peptide with Flanking epitope CxxC/CxxS, where x could be any amino acid, and phosphoserine (PS).

In some embodiments, the immunosuppressive moiety reduces inflammation in the subject. In some embodiments, moieties that reduce inflammation are those that reduce or inhibit the immune system inflammatory response. In some embodiments, the immunosuppressive moiety comprises inflammation reducing moieties including for example Z2-Y12, Z1-Y15 and Z1-Y19 (See Nat Biotechnol. 2016 March; 34(3): 345-352., which is incorporated by reference herein).

In some embodiments, the immunosuppressive moiety comprises moieties that target the secondary lymphoid organs and the liver. As used herein, “secondary lymphoid organs” refers to the organs of the lymphatic system that maintain mature naive lymphocytes and initiate an adaptive immune response. In some embodiments, the secondary lymphoid system comprises lymph nodes and the spleen. In some embodiments, the immunosuppressive moiety that targets the secondar lymph organs and the liver are N-acetylgalactosamine (GalNAc), N-Acetylglucosamine (GlcNAc), N-acetylneuraminic acid (NeuAc or sialic acid), galactose, and fucose, and combinations thereof. In a particular embodiment, the immunosuppressive moiety that targets the secondar lymph organs and the liver is phosphoserine (PS). In some embodiments, the immunosuppressive moiety comprises a combination of N-acetylgalactosamine (GalNAc), N-Acetylglucosamine (GlcNAc), N-acetylneuraminic acid (NeuAc or sialic acid), galactose, fucose, and PS.

In a particular embodiment, the ISM is a phosphoserine (PS) moiety. The PS moiety is or includes the following structure:

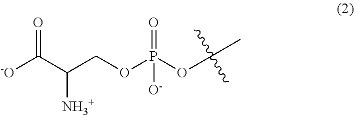

wherein the wavy line indicates a bond to a linker or a direct bond to the viral vector.

In another embodiment, the ISM is a polysialic acid (PSA) moiety. PSA is or includes the following structure:

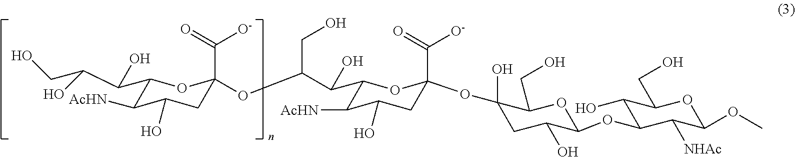

wherein Ac represents acetyl; and n is at least 2. The right-most bond in the above structure indicates a bond to a linker or a direct bond to the viral vector. In different embodiments, n is a value of precisely or at least, for example, 2, 5, 10, 20, 30, 40, 50, 100, 200, 300, 400, or 500, or a value within a range bounded by any two of the foregoing values (e.g., 2-500, 2-200, 2-100, 2-50, 2-20, 10-500, 10-200, 10-100, 10-50, or 10-20).

In some embodiments, the immunosuppressive moiety comprises a class of compounds known as mTOR inhibitors. In such embodiments, the immunosuppressive moiety may comprise one or more mTOR inhibitors. In some embodiments, the mTOR inhibitor is Rapamycin, Temsirolimus, Everolimus, and Umirolimus. In some embodiments, the immunosuppressive moiety comprises a combination of the listed mTOR inhibitors.

In some embodiments, the immunosuppressive moiety (ISM) is selected from small molecules, polymeric molecules, and peptides, wherein the small molecules, polymeric molecules, and peptides typically have a molecular weight of at least 100 g/mol, 200 g/mol, or 500 g/mol, 1000 g/mol, 2000 g/mol, and up to 5,000 g/mol, 10,000 g/mol, 20,000 g/mol, 50,000 g/mol, or 100,000 g/mol (e.g., 100-50,000 g/mol, 100-10,000 g/mol). In some embodiments, the immunosuppressive moiety (ISM) has a molecular weight not more than 50,000 g/mol. In some embodiments, the immunosuppressive moiety (ISM) has a molecular weight not more than 30,000 g/mol. In some embodiments, the immunosuppressive moiety (ISM) has a molecular weight not more than 20,000 g/mol. In some embodiments, the immunosuppressive moiety (ISM) has a molecular weight not more than 10,000 g/mol. In some embodiments, the immunosuppressive moiety (ISM) has a molecular weight of between 500 g/mol to 50,000 g/mol. In some embodiments, the immunosuppressive moiety (ISM) has a molecular weight of between 1000 g/mol to 30,000 g/mol. In some embodiments, the ISM has a molecular weight of between 5000 g/mol to 20,000 g/mol.

In some embodiments, the immunosuppressive moiety comprises molecules from apoptotic cells. Examples of molecules from apoptotic cells are, but are not limited to, phosphatidylserine and chromatin oligonucleotide.

In some embodiments, the immunosuppressive moiety comprises molecules from a spleenocyte.

It is noted that the immunosuppressive moiety can be selected from one or more of the listings above. Additionally, the immunosuppressive moiety categorized listings are not limiting. A member of one of the listed categories above can be a member of other listed categories above and are not static in their category placement.

Structures of some exemplary immunosuppressant moieties are shown below:

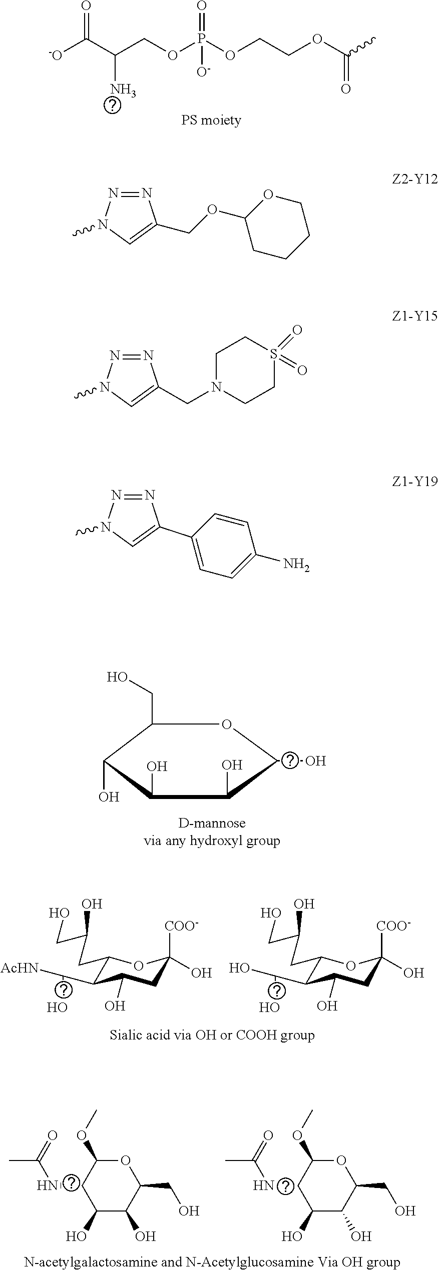

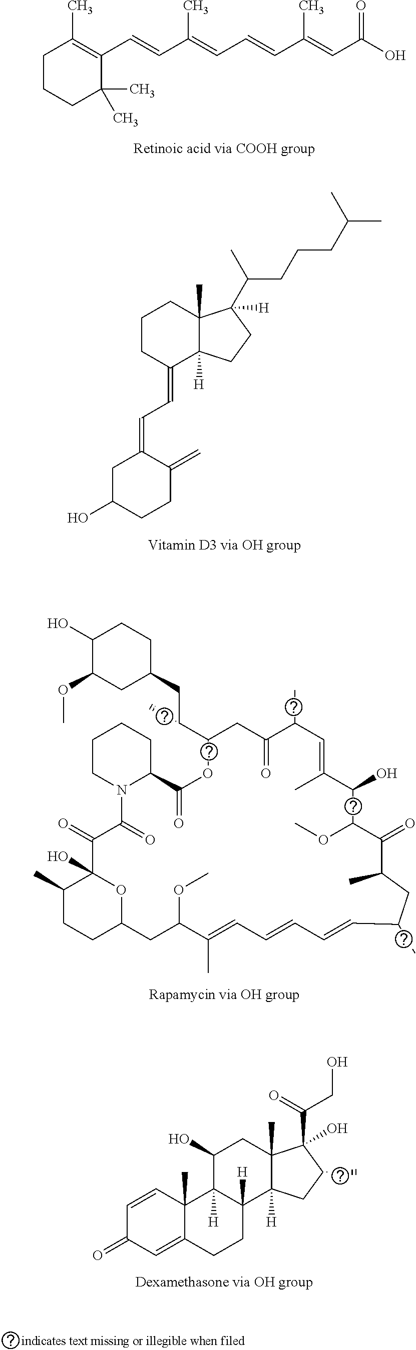

### Linkage Between Viral Vector and Immunosuppressive Moiety

In one set of embodiments, the viral vector (VV) is directly attached to the immunosuppressive moiety (ISM), i.e., without a linker. Direct attachment can be achieved by, for example, reacting one or more groups native to the ISM with one or more groups native to the VV to form one or more covalent bonds, ionic bonds, or hydrogen bonds between the ISM and VV. Groups native to the VV may include those found in proteins or lipids, e.g., amino (or ammonium), thiol, carboxylic acid, and hydroxy groups. Groups native to the ISM may include, for example, amino group (e.g., in the case of the ISM being a phosphoserine or polysialic acid), carboxylic acid group (e.g., retinoic acid), or hydroxy group (e.g., N-acetylgalactosamine). For example, a native carboxylic acid group of the ISM may be activated by methods well known in the art to react and form an amide bond with a native amino group of the VV. In some embodiments, groups native to the VV are provided by one or more molecules on the surface of a viral vector, such as proteins or lipids on the surface of a virus (e.g., proteins or lipids of the capsid or envelope of a virus), in which case, an immunosuppressive moiety can attach to a surface site of a VV. As used herein, the term “surface site” refers to a component or molecule on the surface of a viral vector, such as a protein or a lipid (e.g., proteins or lipids of the capsid or envelope of a virus), that provides one or more reactive groups for forming covalent linkage. Examples of molecules of a viral vector that can provide a surface site include but not limited to capsid proteins, gag proteins, envelope proteins, and/or lipids.

In another set of embodiments, the VV is indirectly attached to the ISM via a linker (L), which may be linear or branched, as further described below. In some embodiments, the ISM is covalently linked through a linker to a surface site of the VV (i.e., a molecule component on the surface of the VV). The linker (L), if present, may be or include a peptide, saccharide, lipid, or non-biological molecule or polymer. In some embodiments, L is a short linker, which may correspond to a length of no more than or less than 1.5 nm, 1.0 nm, or 0.5 nm (15, 10, or 5 Å, respectively). In other embodiments, L is a long linker, which may correspond to a length of at least or above 1.5 nm, 2 nm, 3 nm, 4 nm, 5 nm, 10 nm, 15 nm, 20 nm, 30 nm, 40 nm, 50 nm, or 100 nm. The length may also be within a range bounded by any two of the foregoing values, e.g., 0.5-100 nm, 1-100 nm, 2-100 nm, 0.5-50 nm, 1-50 nm, 2-50 nm, 0.5-10 nm, 1-10 nm, or 2-10 nm).

In a first set of embodiments, the linker (L) is or includes a peptide. The term “peptide,” as used herein, is meant to include single amino acids (monopeptides), dipeptides, tripeptides, oligopeptides, and polypeptides. The peptide may be constructed of a single type or different types of amino acids. The amino acid(s) may be selected from any of the well-known essential amino acids. In particular embodiments, the peptide linker is or includes one or more lysine units. In some embodiments, the peptide linker is or includes a polylysine block, which may contain at least 2, 5, or 10 lysines and up to 15, 20, 25, 30, 40, or 50 lysines. In some embodiments, the peptide linker contains no more than 20 or 25 amino acid (or more specifically, lysine) units (e.g., 2-20 or 2-25 units).

In a second set of embodiments, the linker (L) is or includes a saccharide. The term “saccharide,” as used herein, is meant to include single saccharides (monosaccharides), disaccharides, trisaccharides, oligosaccharides, and polysaccharides. The saccharide may be constructed of a single type or different types of saccharide units. The saccharide(s) may be selected from any of the known types of saccharides, such as glucose, fructose, and galactose, as well as amino-functionalized versions thereof (e.g., glucosamine and galactosamine) and N-acetyl functionalized versions thereof (e.g., N-acetyl glucosamine). In some embodiments, the saccharide contains no more than or less than 5, 10, 20, 25, 30, 40, or 50 saccharide units.

In a third set of embodiments, the linker (L) is or includes a lipid. The lipid may be any of the lipids known in the art. As well know, the lipid moiety is constructed of a polyol portion (e.g., a diol, glycerol, phosphatidylglycerol, phosphatidylethanolamine, or phosphatidylserine) that has been esterified with one or two fatty acid molecules to result in a monoacyl or diacyl lipid, wherein the term “acyl” refers to a RC(═O) group in which R is a linear or branched hydrocarbon (fatty) chain containing at least eight and typically up to 30 carbon atoms, wherein the hydrocarbon chain may be saturated or contain one or more carbon-carbon double bonds. The lipid moiety may be, for example, a diacyldiol (e.g., diacylethyleneglycol), diacylglycerol (diacylglyceride), diacylphosphatidylglycerol, diacylphosphatidylethanolamine, or diacylphosphatidylserine moiety. The acyl (i.e., “fatty acyl”) portion may be derived from any of the known fatty acids. Some examples of fatty acyl portions include oleoyl, palmitoyl, lauryl, myristoyl, stearoyl, linoleoyl, and arachidonyl.

In a fourth set of embodiments, the linker (L) is or includes a non-biological molecule or polymer. The non-biological molecule may be or include, for example, a linear or branched alkylene linker, bifunctional crosslinker, aromatic group (e.g., phenylene), or combination thereof. The non-biological polymer may be any of the polymers known in the art compatible with living organisms, such as polyethylene oxide, polyamine, polyamide, polyurea, and polyester.

In some embodiments, any one or more classes or specific types of linkers described above is excluded from the linker (L).

As the linker (L) may be absent or present (i.e., optional), the modified viral vector can be expressed by the following structure:

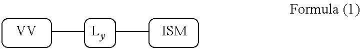

In Formula (1) above, VV is the viral vector, such as any of the viral vectors described above; L is a linker, wherein y is 0 or 1, which corresponds to the absence or presence of the linker, respectively; ISM is an immunosuppressive moiety, such as any of the ISMs described above; and z is at least 1. The lines connecting VV, L_{y}, and ISM represent covalent bonds. In some embodiments, the linker or ISM is attached to the viral vector via an amino group of the viral vector, wherein the amino group may be on a protein or lipid molecule at the surface of the viral vector (e.g., a protein or lipid of a capsid or envelope of a virus). Although Formula (1) depicts an embodiment of a single L_{y}-(ISM)_{z} moiety, this embodiment is for illustration and not intended to be limiting. In some embodiments, a multiplicity of L_{y}-(ISM)_{z} moieties (i.e., either a multiplicity of L-(ISM)_{z} or ISM, depending on y) are attached to the VV. As indicated by the variable z optionally being greater than 1, a single L may attach to more than one ISM. A single L can attach to more than one ISM by having one or more branch points in L, each of which can attach to an ISM. In this way, the variable z can have a value of precisely or at least, for example, 2, 3, 4, 5, 6, 7, 8, 9, 10, 11, 12, 15, 18, 20, 22, 25, or 30, or a value within a range bounded by any two of the foregoing values, for a single L.

In some embodiments, the linker (L) is present, in which case the modified viral vector is or includes the following structure:

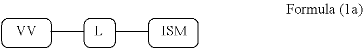

In some embodiments of Formula (1a), the linker (L) is attached to the viral vector (VV) via an amino group of the VV. The amino group may reside on a molecule at the surface (e.g., a protein or lipid of the capsid or envelop) of the VV. Although Formula (1a) depicts an embodiment of a single L-(ISM)_{z} moiety, this embodiment is for illustration and not intended to be limiting. Typically, a multiplicity of L-(ISM)_{z} moieties are attached to the VV. The multiplicity (density) of L-(ISM)_{z} moieties on the VV may correspond to, for example, at least or up to 10%, 20%, 30%, 40%, 50%, 60%, 70%, 80%, or 90% of native groups (e.g., amino groups) of the VV being attached to the L-(ISM)_{z} moiety. Moreover, as indicated by the variable z optionally being greater than 1, a single L may attach to more than one ISM. A single L can attach to more than one ISM by having one or more branch points in L, each of which can attach to an ISM. In this way, the variable z can have a value of precisely or at least, for example, 2, 3, 4, 5, 6, 7, 8, 9, 10, 11, 12, 15, 18, 20, 22, 25, or 30, or a value within a range bounded by any two of the foregoing values, for a single L. Notably, a single L may attach to the VV by more than one bond, and the single L may attach to one or more ISMs. As further discussed below, L in Formulas (1) and (1a) includes the possibility of reactive functional groups linking between VV and L and/or between L and ISM.

In some embodiments, more than one ISM is covalently bound to the VV. In some embodiments, a multiplicity of L-ISM moieties are attached to the VV, wherein each L-ISM moiety contains a single ISM. In other embodiments, a multiplicity of L-(ISM)_{z} moieties are attached to the VV, wherein each L-(ISM)_{z} moiety contains a branched L attached to more than one ISM (i.e., wherein z is at least 2). In some embodiments, 1-10,000 ISMs are covalently bound to the VV either as L-ISM or L-(ISM)_{z} moieties. In other embodiments, 1-5,000 ISMs are covalently bound to the VV. In other embodiments, 1-2,000 ISMs are covalently bound to the VV. In other embodiments, 100-2,000 ISMs are covalently bound to the VV. In some embodiments, ISMs are first attached to a linker before being attached to the VV. In some embodiments, the linkers are first attached to the VV followed by attachment of ISMs to the linkers. In each case, a single linker may have one or a multiplicity (e.g., 2, 5, 10, 20, 30, 40, or 50, or range therein) of ISMs. The single linker may include a multiplicity of ISMs by having branched portions, each connecting the main linker portion to the ISM. In the case of a linker attached to a single ISM, the linker may be linear (i.e., not contain any branching portions), wherein the ISM may be bound to any part of the linear linker, typically the terminal end of the linker (i.e., farthest from the VV).

In some embodiments, the modified viral vector is or includes the following structure:

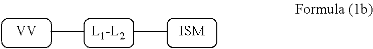

In Formula (1b), L₁ and L₂ are portions of the linker. L₁ represents a bifunctional crosslinker (containing two reactive functional groups), such as one possessing an amino-reactive and thiol-reactive group, wherein the amino reactive group is bound to (i.e., has reacted with) an amino group of the viral vector (VV). L₂ represents a linking portion containing a thiol group bound to (i.e., reacted with) the thiol-reactive group of L₁, wherein L₂ is also bound to the ISM. Thus, L₁ includes the reaction products resulting from reaction of its amino-reactive and thiol-reactive groups with amino group of VV and thiol group of L₂, respectively. L₂ may be any of the peptides, saccharides, lipids, or non-biological molecules or polymers described earlier above, provided it contains a thiol group bound to the thiol-reactive group of L₁. Although Formula (1b) depicts an embodiment of a single L₁-L₂-(ISM)_{z} moiety, this embodiment is for illustration and not intended to be limiting. Typically, a multiplicity of L₁-L₂-(ISM)_{z} moieties are attached to the VV. The multiplicity (density) of L₁-L₂-(ISM)_{z} moieties on the VV may correspond to, for example, at least or up to 10%, 20%, 30%, 40%, 50%, 60%, 70%, 80%, or 90% of native groups (e.g., amino groups) of the VV being attached to the L₁-L₂-(ISM)_{z} moiety.

In an alternative embodiment of Formula (1b), the viral vector (VV) is modified to contain a thiol group, and L₁ represents a thiol-reactive group bound to (i.e., has reacted with) the thiol group of VV, wherein L₂ is bound to L₁ and the ISM. L₂ may be any of the peptides, saccharides, lipids, or non-biological molecules or polymers described earlier above.

In another alternative embodiment of Formula (1b), VV and L₂ are modified to contain an azide or alkyne group in order for VV and L₂ to attach by azide-alkyne cycloaddition click chemistry. L₁ represents or includes a 1,2,3-triazole group connecting VV and L₂, wherein the 1,2,3-triazole group is the result of an azide-alkyne cycloaddition click chemistry reaction between the azide or alkyne group on the VV and alkyne or azide group, respectively, on L₂. L₂ may be any of the peptides, saccharides, lipids, or non-biological molecules or polymers described earlier above.

In another alternative embodiment of Formula (1b), L₁ and ISM are modified to contain an azide or alkyne group in order for L₁ and ISM to attach by azide-alkyne cycloaddition click chemistry. L₂ represents or includes a 1,2,3-triazole group connecting L₁ and ISM, wherein the 1,2,3-triazole group is the result of an azide-alkyne cycloaddition click chemistry reaction between the azide or alkyne group on L₁ and alkyne or azide group, respectively, on ISM. L₁ may be any of the peptides, saccharides, lipids, or non-biological molecules or polymers described earlier above.

In any of the above-described embodiments of Formula (1b), a multiplicity of L-(ISM)_{z} moieties are typically attached to the VV. The multiplicity of L-(ISM)_{z} moieties may correspond to, for example, at least or up to 10%, 20%, 30%, 40%, 50%, 60%, 70%, 80%, or 90% of native groups (e.g., amino groups) of the VV being attached to the L-(ISM)_{z} moiety. Moreover, as indicated by the variable z optionally being greater than 1, a single L may attach to more than one ISM. A single L can attach to more than one ISM by having one or more branch points in L, each of which can attach to an ISM. In this way, the variable z can have a value of precisely or at least, for example, 2, 3, 4, 5, 6, 7, 8, 9, 10, 11, 12, 15, 18, 20, 22, 25, or 30, or a value within a range bounded by any two of the foregoing values, for a single L. Notably, a single L may attach to the VV by more than one bond, and the single L may attach to one or more ISMs.

In some embodiments, the modified viral vector is or includes the following structure:

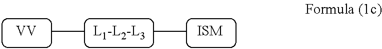

In Formula (1c), L₁, L₂, and L₃ are portions of the linker. L₁ represents a bifunctional crosslinker, such as one possessing an amino-reactive and thiol-reactive group, wherein the amino reactive group is bound to (i.e., has reacted with) an amino group of the viral vector. L₂ represents a linking portion containing a thiol group bound to the thiol-reactive group of L₁, wherein L₂ may be any of the peptides, saccharides, lipids, or non-biological molecules or polymers described earlier above. L₃ represents a bifunctional crosslinker possessing an amino-reactive and thiol-reactive group, wherein the amino reactive group is bound to (i.e., has reacted with) an amino group of L₂ and the thiol-reactive group is bound to (i.e., has reacted with) a thiol group of the ISM, or alternatively, the amino reactive group is bound to (i.e., has reacted with) an amino group of the ISM and the thiol-reactive group is bound to (i.e., has reacted with) a thiol group of L₂. Although Formula (1c) depicts an embodiment of a single L₁-L₂-L₃-(ISM), moiety, this embodiment is for illustration and not intended to be limiting. Typically, a multiplicity of L₁-L₂-L₃-(ISM)_{z} moieties are attached to the VV. The multiplicity (density) of L₁-L₂-L₃-(ISM)_{z} moieties on the VV may correspond to, for example, at least or up to 10%, 20%, 30%, 40%, 50%, 60%, 70%, 80%, or 90% of native groups (e.g., amino groups) of the VV being attached to the L₁-L₂-L₃-(ISM)_{z} moiety.

In alternative embodiments of Formula (1c), the viral vector (VV) is modified to contain a thiol group, and L₁ represents a thiol-reactive group bound to the thiol group of VV. L₃ represents a bifunctional crosslinker possessing an amino-reactive and thiol-reactive group, wherein the amino reactive group is bound to (i.e., has reacted with) an amino group of L₂ and the thiol-reactive group is bound to (i.e., has reacted with) a thiol group of the ISM, or alternatively, the amino reactive group is bound to (i.e., has reacted with) an amino group of the ISM and the thiol-reactive group is bound to (i.e., has reacted with) a thiol group of L₂. L₂ may be any of the peptides, saccharides, lipids, or non-biological molecules or polymers described earlier above.

In another alternative embodiment of Formula (1c), VV and L₂ are modified to contain an azide or alkyne group in order for VV and L₂ to attach by azide-alkyne cycloaddition click chemistry. L₁ represents or includes a 1,2,3-triazole group connecting VV and L₂, wherein the 1,2,3-triazole group is the result of an azide-alkyne cycloaddition click chemistry reaction between the azide or alkyne group on the VV and alkyne or azide group, respectively, on L₂. L₂ may be any of the peptides, saccharides, lipids, or non-biological molecules or polymers described earlier above. L₃ is a linking portion attaching L₂ and ISM. L₃ may be, for example, the result of reacting an amino group on L₂ with an amino-reactive group on ISM, or the result of reacting an amino-reactive group on L₂ with an amino group on ISM, or the result of reacting a thiol group group on L₂ with a thiol-reactive group on ISM, or the result of reacting a thiol-reactive group on L₂ with a thiol group on ISM, or L₃ may be a 1,2,3-triazole group resulting from a cycloaddition reaction between an alkynyl group on L₂ with an azide group on ISM, or L₃ may be a 1,2,3-triazole group resulting from a cycloaddition reaction between an alkynyl group on ISM and an azide group on L₂.

In another alternative embodiment of Formula (1c), L₂ and ISM are modified to contain an azide or alkyne group in order for L₂ and ISM to attach by azide-alkyne cycloaddition click chemistry. L₃ represents or includes a 1,2,3-triazole group connecting L₂ and ISM, wherein the 1,2,3-triazole group is the result of an azide-alkyne cycloaddition click chemistry reaction between the azide or alkyne group on L₂ and alkyne or azide group, respectively, on ISM. L₂ may be any of the peptides, saccharides, lipids, or non-biological molecules or polymers described earlier above, provided it is modified to contain an azide or alkyne group. L₁ is a linking portion attaching L₂ and VV. L₁ may be, for example, the result of reacting an amino group on L₂ with an amino-reactive group on VV, or the result of reacting an amino-reactive group on L₂ with an amino group on VV, or the result of reacting a thiol group group on L₂ with a thiol-reactive group on VV, or the result of reacting a thiol-reactive group on L₂ with a thiol group on VV, or L₁ may be a 1,2,3-triazole group resulting from a cycloaddition reaction between an alkynyl group on L₂ with an azide group on VV, or L₁ may be a 1,2,3-triazole group resulting from a cycloaddition reaction between an alkynyl group on VV and an azide group on L₂.

In any of the above-described embodiments of Formula (1c), a multiplicity of L-(ISM)_{z} moieties are typically attached to the VV. The multiplicity of L-(ISM)_{z} moieties may correspond to, for example, at least or up to 10%, 20%, 30%, 40%, 50%, 60%, 70%, 80%, or 90% of native groups (e.g., amino groups) of the VV being attached to the L-(ISM)_{z} moiety. Moreover, as indicated by the variable z optionally being greater than 1, a single L may attach to more than one ISM. A single L can attach to more than one ISM by having one or more branch points in L, each of which can attach to an ISM. In this way, the variable z can have a value of precisely or at least, for example, 2, 3, 4, 5, 6, 7, 8, 9, 10, 11, 12, 15, 18, 20, 22, 25, or 30, or a value within a range bounded by any two of the foregoing values, for a single L. Notably, a single L may attach to the VV by more than one bond, and the single L may attach to one or more ISMs.

In some embodiments, the ISM is specifically phosphoserine (PS). In such embodiments, the modified viral vector can have any of the following structures:

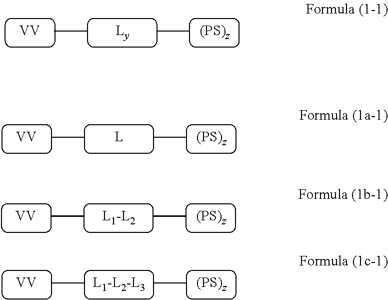

In the above Formulas (1-1), (1a-1), (1b-1), and (1c-1), VV, L, y, L₁, L₂, and L₃ are as described above under Formulas (1), (1a), (1b), and (1c), including all exemplary embodiments provided therein, including the possibility for L or sub-linker component thereof (e.g., L₁, L₂, or L₃) to have one or more branch points, thereby permitting a single linker to attach to more than one or a multiplicity of ISMs.

In some embodiments, the ISM is specifically polysialic acid (PSA). In such embodiments, the modified viral vector can have any of the following structures:

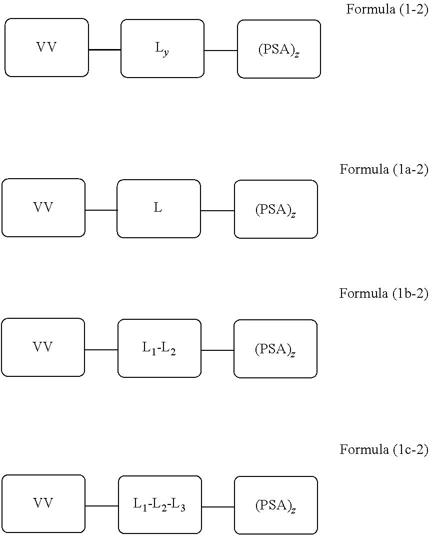

In the above Formulas (1-2), (1a-2), (1b-2), and (1c-2), VV, L, y, L₁, L₂, and L₃ are as described above under Formulas (1), (1a), (1b), and (1c), including all exemplary embodiments provided therein, including the possibility for L or sub-linker component thereof (e.g., L₁, L₂, or L₃) to have one or more branch points, thereby permitting a single linker to attach to more than one or a multiplicity of ISMs.

In embodiments in which L₁ and/or L₃ are present, L₁ and/or L₃ may represent the product of reaction between any two reactive functional groups, such as between an amine and amine-reactive group, or between a thiol and thiol-reactive group, or between alkyne and azide reactive groups. Notably, in any of the formulas disclosed above in which L₁ and/or L₃ represents a bifunctional crosslinker, L₁ and/or L₃ may possess a combination of reactive functional groups beyond amine-amine, amine-thiol, and alkyne-azide coupling. L₁ and/or L₃ may function, for example, as an amine-carboxy, thiol-carboxy, and thiol-thiol coupler. Some examples of amine-amine bifunctional crosslinkers include bis(NHS)-ester compounds (where NHS=N-hydroxysuccinimide) and sulfonated versions thereof, as well as di-imidoester compounds, many of which are commercially available. Some examples of amine-thiol bifunctional crosslinkers include NHS-maleimido compounds and their sulfonated versions, many of which are commercially available. Some examples of thiol-thiol bifunctional crosslinkers include bis(maleimide) compounds and their sulfonated versions, as well known in the art. Some examples of amine-carboxy bifunctional crosslinkers include the carbodiimides, such as N,N′-dicyclohexylcarbodiimide (DCC), as well known in the art. L₁ and/or L₃ may independently be selected from any of the foregoing bifunctional crosslinkers in any of the formulas provided in this disclosure. As understood, although L₁ and/or L₃ may be identified as bifunctional crosslinkers, L₁ and/or L₃ are present in any of the formulas above as reaction products resulting from reaction between reactive groups of the bifunctional crosslinker and groups present on VV, L₂, or ISM.

In some embodiments, the crosslinker compound comprises a N-hydroxysuccinimide ester-maleimide heterobifunctional aliphatic reagent for crosslinking an amino group with a thiol group. Some examples of crosslinker compounds include AMAS, BMPS, GMBS, Sulfo-GMBS, MBS, Sulfo-MBS, SMCC, Sulfo-SMCC, EMCS, Sulfo-EMCS, SMPB, Sulfo-SMPB, SMPH, LC-SMCC, and Sulfo-KMUS.

In the case of a branched linker, the branched linker may have the following exemplary structure:

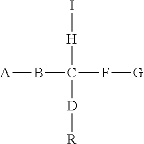

In the above branched linker structure, C is a branching point of the linker, and B, D, F, and H are branching portions, each of which terminates in a reactive functional group (A, R, G, and I, respectively) which in turn attach to the viral vector (VV) and ISM, as further discussed below. At least one of the reactive functional groups (A, R, G, and I) connects to the VV, and at least one of the reactive functional groups (A, R, G, and I) connects to the ISM. In some embodiments, the VV becomes attached to a multiplicity of L by reaction of reactive groups on VV (e.g., “R₁”) with reactive functional groups R on each linker to form the following linkage: (VV)-R₁-R-L-(ISM)_{z}, wherein R₁-R represents the product of reaction between R₁ and R, and L may link to more than one ISM via its remaining A, G, and/or I reactive functional groups. In other embodiments, the VV becomes attached to L by more than one attachment point (e.g., two or three of A, R, G, and I reactive functional groups), and each L may link to one or more ISM.

In some embodiments, the reactive functional groups (e.g., A, G, R, R₁ and I) present in the branched linker or in any of the formulas provided in this disclosure are independently selected from H, F, Cl, Br, I, SH, protected thiols, NH₂, —NH— (secondary amine), N═C═O, protected NCO, N═C═S, COOH, activated ester, aldehyde, COSH, C(═S)SH, OCOOH, OCOSH, OC(═S)OH, SC(═O)SH, SC(═S)SH, N(C═O)NH2, N(C═NH)NH2, N(C═S)NH2, δ-valerolactone, ε-caprolactone, CH2=CH—C(═O)—O—, CH2=CH—C(═O)—NH—, CH2=CH—C(═O)—S—, CN, CH2=C(CH3)-C(═O)—O—, CH2=C(CH3)-C(═O)—NH—, OH, azides, alkynes, C6-C10 aryl groups, cyclic groups (isobornyl, cyclohexyl, cyclopentyl), and fluoro (perfluorobutyl, perfluoroethyl) derivatives.

In some embodiments, B, F and H in the branched linker are selected independently from —(CH2)x-, where x is an integer from 0 to 20.

In some embodiments, C in the branched linker can be carbon, nitrogen, or silicon.

In some embodiments, D in the branched linker is C(═O)(CH₂)x or —(CH₂)x-, where x is an integer from 1 to 20.

In some embodiments, the modified viral vector disclosed herein achieves a transfection efficiency that is at least 30% of the transfection efficiency by an unmodified viral vector. In other embodiments, the modified viral vector disclosed herein achieves a transfection efficiency that is at least 40% of the transfection efficiency by an unmodified viral vector. In other embodiments, the modified viral vector disclosed herein achieves a transfection efficiency that is at least 50% of the transfection efficiency by an unmodified viral vector. In other embodiments, the modified viral vector disclosed herein achieves a transfection efficiency that is at least 60% of the transfection efficiency by an unmodified viral vector. In other embodiments, the modified viral vector disclosed herein achieves a transfection efficiency that is at least 70% of the transfection efficiency by an unmodified viral vector.

### Compositions and Methods for Introducing a Modified Viral Vectors Into a Cell or a Subject

Gene therapy via administration of virus vectors provides a promising approach to treat a variety of inherited and acquired diseases. However, vector immunogenicity caused by a first administration of a virus vector can limit subsequent administrations of viral vectors. In some cases, persistent high-titer antibodies may be triggered by multiple vector administrations reducing any benefit of repeated viral vector-based treatments. In some embodiments, the disclosed compositions and methods may reduce the immunogenicity of viral vectors thereby enabling multiple administrations of viral-based gene delivery.

In some embodiments, this disclosure provides a method of introducing a viral vector to a subject by introducing a modified viral vector disclosed herein, for example, to treat and/or prevent a disease.

In some embodiments, a modified viral vector is transfected into the cells of a subject ex vivo and the resulting cells are then infused in vivo. In some embodiments, the method comprising contacting the cells multiple times with a modified viral vector. In some embodiments, the method comprising contacting the cells multiple times, each time with multiple modified viral vectors.

In some embodiments, a modified viral vector is administered directly in vivo and delivered into the subject's cells in vivo.

In some embodiments, the modified viral vector is administered through enteral routes of administration. In some embodiments, the modified viral vector is administered through parenteral routes of administration. In some embodiments, the modified viral vector is administered orally. In some embodiments, the modified viral vector is administered sublingually. In some embodiments, the modified viral vector is administered rectally. In some embodiments, the modified viral vector is administered through inhalation by powder aerosols. In some embodiments, the modified viral vector is administered through inhalation by pressurized metered-dose aerosols comprising the modified viral vector in liquefied inert propellant. In some embodiments, the modified viral vector is administered subcutaneously. In some embodiments, the modified viral vector is administered intramuscularly. In some embodiments, the modified viral vector is administered intradermally. In some embodiments, the modified viral vector is administered intravenously. In some embodiments, the modified viral vector is administered intra-arterially. In some embodiments, the modified viral vector is administered intrathecally. In some embodiments, the modified viral vector is administered intraperitoneally. In some embodiments, the modified viral vector is administered intravitreally.

In some embodiments, a subject administered with a modified viral vector as described in the disclosure exhibits a reduced immune response as compared to a control subject administered an unmodified viral vector.

In some embodiments, the method comprises administering a single modified viral vector of the disclosure to a subject multiple times. In some embodiments, the method comprises administering more than one modified viral vectors of the disclosure to a subject at multiple times. In some embodiments, the method comprises administering more than one modified viral vectors of the disclosure to a subject at multiple times, wherein the subject exhibits a reduced immune response as compared to a control subject administered an unmodified viral vector at multiple times.

In some embodiments, the method comprises administering to a subject with (i) a modified viral vector described in the disclosure at a first point in time and subsequently (ii) administered the modified viral vector at a second point in time, and the subject exhibits a reduced immune response as compared to a control subject administered an unmodified viral vector at the first and second points in time. In some embodiments, the method comprises administering to a subject with (i) a modified viral vector described in the disclosure at a first point in time, and subsequently (ii) a different modified viral vector described in the disclosure at a second point in time; and the subject exhibits a reduced immune response as compared to a control subject administered an unmodified viral vector at two the first and second points in time.

In some embodiments, the second point in time is between 1 day and 49 days after the first point in time. In some embodiments, the second point in time is between 1 day and 49 days after the first point in time. In some embodiments, the second point in time is between 3 days and 35 days after the first point in time. In some embodiments, the second point in time is between 7 days and 28 days after the first point in time. In some embodiments, the second point in time is between 14 days and 21 days after the first point in time. In some embodiments, the second point in time is at least 24 hours after the first point in time. In some embodiments, the second point in time is at least 72 hours after the first point in time. In some embodiments, the second point in time is at least 7 days after the first point in time. In some embodiments, the second point in time is at least 14 days after the first point in time. In some embodiments, the second point in time is at least 21 days after the first point in time.

Immunogenicity of a viral vector can be determined based on a variety of methods described in the art. These methods include a variety of functional assays, such as antibody enzyme-linked immunosorbent assay (ELISA), enzyme-linked immunospot (ELISPOT), delayed-type hypersensitivity, tetramer analysis, flow cytometry-based analysis of cytokine expression, neutralizing antibody assay, cytometry by time of flight (CyTOF), and PCR-based detection of T-cell receptor gene usage or cytokine production. (Hui D. J., et al., AAV capsid CD8⁺ T-cell epitopes are highly conserved across AAV serotypes. Mol. Ther. Methods Clin. Dev. 2015; 2:15029.; Martino A. T., et al., Measuring immune responses to recombinant AAV gene transfer. Methods Mol. Biol. 2011; 807:259-272; Veron P., et al., Humoral and cellular capsid-specific immune responses to adeno-associated virus type 1 in randomized healthy donors. J. Immunol. 2012; 188:6418-6424; and Kuranda K., et al., Exposure to wild-type AAV drives distinct capsid immunity profiles in humans. J. Clin. Invest. 2018; 128:5267-5279, all of which are incorporated by reference herein as they pertain to immunogenicity assays; see also Example 7 of this disclosure, below).

In some embodiments reduced immunogenicity can be determined based on analysis of T cell functions, such as analysis of Treg activation and/or measurement of the amount of anti-vector antibodies as compared to a control. Assays for analyzing T cell functions are known in the art and include, but are not limited to, cytokine-based functional assays, HLA class Pepitope tetrameric complexes, and PCR-based methods. T cell function can also be measured through AAV8-specific mouse Interferon gamma ELISPOT and Anti-AAV8 IgG secreting B cell ELISPOT with commercially available kits as described in Example 7 below. Regulatory T cells (Treg) activation can be measured through methods known in the art, including but not limited to, the suppression assay, which measures the ability of Tregs to suppress T cell proliferation, measuring expression of CCR5, measuring FOXP3 demethylation including qPCR and epigenetic sequencing methylation analysis of Sanger sequencing traces. Additionally, as shown below in the Examples, Tregs can be stained with antibodies and analyzed by flow cytometry. Antibodies to viral vectors can be examined through methods known in the art. Such methods include ELISA and flow cytometry through the use of anti-viral vector antibodies through commercially available kits, and as seen in Example 7 below.

In some embodiments, a modified viral vector is used for multiple in vitro and in vivo applications.

In some embodiments, a modified viral vector is used for clustered regularly interspaced short palindromic repeats-Cas endonuclease (CRISPR-Cas) gene editing in vitro and in vivo.

In some embodiments, a modified viral vector is administered to a subject to treat or prevent a condition or disease in a subject, including, but not limited to, an infectious disease (e.g., infection caused by coronavirus including for example SARS-CoV-2), an autoimmune disease, or cancer. In some embodiments, a modified viral vector is delivered along with checkpoint inhibitor (e.g., anti-Programmed death-ligand 1 (anti-PD-L1) antibody, anti-cytotoxic T-lymphocyte-associated protein 4 (anti-CTLA4) to treat cancer.

The term “treatment” or “treating” refers to preventing or delaying the onset, slowing down the progression, and/or or ameliorating the symptoms of the disorder.

The term “subject” refers to any mammalian subject, including human.

A modified viral vector can be delivered “naked” by direct injection into the blood stream or the desired tissue or organ of a subject. Alternatively, a modified viral vector can be combined with a lipid compound which facilitates the uptake of the molecule by cells. The lipid compound include liposome, lipofectins, cytofectins, lipid-based positive ions, and then introduced into the body fluids, the blood stream, or a selected tissue site.

A modified viral vector can be administered to a subject through various suitable routes of administration, including the oral, ophthalmic, nasal, topical, transdermal, parenteral (e.g., intravenous, intraperitoneal, intradermal, subcutaneous or intramuscular) route.

In some embodiments, this disclosure provides compositions comprising a modified viral vector, suitable for administration to a subject. The compositions can also include a pharmaceutically acceptable carrier.

As used herein, a pharmaceutically acceptable carrier includes any and all solvents, dispersion media, isotonic agents and the like. Except insofar as any conventional media, agent, diluent, or carrier is detrimental to the recipient or to the therapeutic effectiveness of a modified viral vector, its use is appropriate. The carrier can be liquid, semi-solid, e.g., pastes, or solid carriers. Examples of carriers include oils, water, saline solutions, alcohol, sugar, gel, lipids, liposomes, resins, porous matrices, binders, fillers, coatings, preservatives and the like, or combinations thereof. A modified viral vector can be combined with a carrier in any convenient and practical manner, e.g., by admixture, solution, suspension, emulsification, encapsulation, absorption and the like, and can be made in formulations such as tablets, capsules, powder, syrup, suspensions that are suitable for injections, implantations, inhalations, ingestions or the like.

In some embodiments, a composition comprising any one of the modified viral vectors described above is a vaccine composition. In some embodiments, the vaccine composition comprises an adjuvant(s). In some embodiments, the vaccine composition comprising a modified viral vector is a vaccine against coronavirus including for example SARS-CoV-2. In some embodiments, this application discloses methods for vaccinating a subject comprising administering to a subject a modified viral vector described above.

### EXAMPLES

The steps of the method described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. The examples are not intended to be limiting in any manner.

### Example 1. Synthesis of Immunosuppressive PS Moiety

### Scheme 1. Synthesis of PS Moiety.

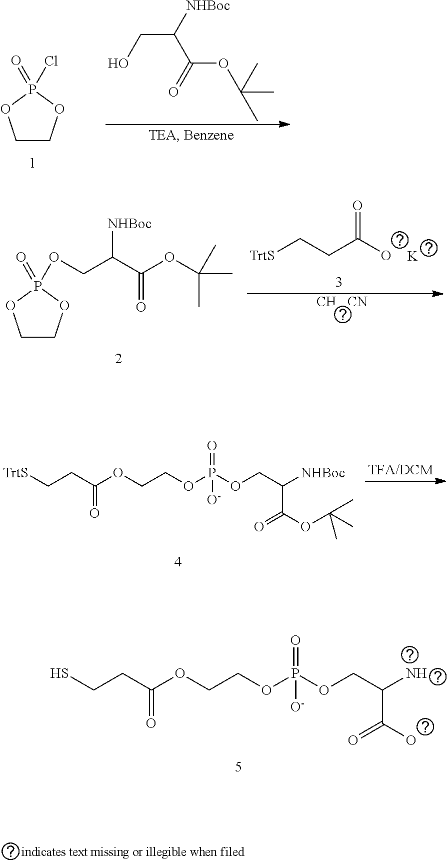

Synthesis of Compound 4: N-Boc-Ser-OtBu (1.0 gm, 3.82 mmol) was dissolved in 30 ml dry benzene and the solution was cooled to 0° C. Next, triethylamine (0.62 mL, 4.6 mmol) was added followed by dropwise addition of 2-chloro-2-oxo-1,3,2-dioxaphospholane 1 (0.42 mL, 4.6 mmol) in 10 mL dry benzene over a period of 30 minutes and then the reaction contents were stirred at room temperature for another 3 hours. After completion of reaction, diethyl ether was poured into the reaction mixture and the precipitated trimethylamine hydrochloride was filtered off. The filtrate was then concentrated under reduced pressure to give Compound 2 as an oil which was used in next step without further purification. Compound 2 was redissolved in 30 mL anhydrous acetonitrile and compound 3 (2.2 gm, 5.7 mmol) along with 18-crown-6 ether (0.14 gm, 0.53 mmol) was added to it. The reaction mixture was then stirred at 55° C. for 72 hours. After the reaction, the reactions contents were filtered, concentrated under vacuum and purified by flash column chromatography to give compound 4 in 56% yield. Results were confirmed through 1H NMR spectra of Compound 4. 1H NMR (300 MHz, D2O) δ 4.29-4.19 (m, 6H), 4.02-3.94 (m, 2H), 2.79 (t, J=6.9 Hz, 2H), 2.69-2.61 (m, 2H).

Synthesis of PS-SH (Compound 5): Compound 4 (1.5 gm, 2.14 mmol) was dissolved in 5 mL dichloromethane and 30 mL trifluoroacetic acid was added to it. The reaction contents were stirred for 5 hours. After completion of the reaction, the reaction mixture was concentrated under vacuum to give a thick viscous liquid. The crude product was then crystallized with MeOH:diethyl ether (1:20) to give desired compound 5 as white powder. Results were confirmed through 1H NMR spectra of compound 5. 1H NMR (300 MHz, D2O) δ 4.29-4.19 (m, 6H), 4.02-3.94 (m, 2H), 2.79 (t, J=6.9 Hz, 2H), 2.69-2.61 (m, 2H). m/z: [M]⁻ 316.0

### Example 2. Synthesis of Polymeric Linker, (KK)8 Peptide

Synthesis of Fmoc-Lys(Z)-Lys(Boc)-OH (KK dimer): Fmoc-Lys(Z)-OH (42.5 g, 0.1 mol) and NHS (13.8 g, 0.12 mol) were dissolved in anhydrous acetonitrile (400 mL) under nitrogen. Then DCC (22.7 g, 0.11 mol) was added and the mixture was stirred at room temperature overnight. In a separate round-bottom flask, H-Lys(Boc)-OH (25.0 g, 0.102 mol) was stirred in a mixture of NaHCO3 6.8%/acetonitrile 1:2 (900 mL) and then the mixture was poured dropwise into the former solution. The reaction was kept stirring for 24 h at rt before adjusting the pH to 6 (1 N HCl). All precipitants were filtered, and acetonitrile was evaporated. Aqueous layer was acidified to pH 1 by 12M HCl and extracted with DCM (400 mL for 3 times). All organic layers were combined, washed with water (1×300 mL), and dried over anhydrous Na2SO4. The crude was crashed out in excess hexane and recrystallized twice in an ethyl acetate/hexane solution. The KK dimer was obtained as a white powder. 1H NMR (300 MHz, CDCl₃) δ 7.776-7.685 (d,2H), 7.605-7.522(t,2H), 7.412-7.269 (m,6H), 7.177-7.039 (m,1H), 6.116-5.925 (d, 1H), 5.163-4.981 (m, 3H),4.881-4.462 (dm, 2H), 4.417-4.236 (m,3H),4.214-4.131 (m,1H),3.212-2.969 (m,4H),1.955-1.582 (m,4H), 1.533-1.227 (m,17H). m/z: [M-Boc+2H]⁺=631.3

Synthesis of (KK)8-C-NH2 Polypeptide: (KK)10-C-NH2 was synthesized by Fmoc Solid Phase Peptide Synthesis (SPPS) on Liberty Blue automated microwave assisted peptide synthesizer (CEM). Sequence synthesis scale was set at 2.5 mmol on Rink amide MBHA resin (0.6 meq/g substitution). Deprotection was performed in 20% piperidine/DMF solution with machine default microwave conditions. Coupling reactions were performed in the presence of a 5-fold molar excess of reagents (amino acid/diisopropylcarbodiimide/Oxyma=0.2M/0.5M/1.0M in DMF) by using 2.5 mmol coupling cycle method provided from CEM. Cleavage was performed using 20 ml of cocktail (TFA/phenol/water/thioanisole/EDT; 82.5/5/5/5/2.5) for 180 minutes at room temperature. Following cleavage, (EK)10-C-NH2 was precipitated out and washed with ice-cold anhydrous ethyl ether. m/z: 1116.7²⁺, 1674.4³⁺, [H-(K(Cbz)K)8C(mob)-NH2, 3347.89] Dimethylation of amine groups was prepared by using HCOOH/HCOH reflux. Z groups was then deprotected in 33% HBr in AcOH solution. The peptide was dissolved in 10% acetic acid and freeze-dried. Econo-Pac 10DG Desalting Columns were used to further enhance the purity of the products.

### Example 3. Preparation of KKPS Modified AAV8 and AAV PHP.eb

2 μl AMAS (10 nM in DMSO) was mixed with 250 μl AAV (2E11 vg/ml in pH7.4 PBS) for 2-hours at room temperature. The unreacted AMAS was removed with Amicon® Ultra Centrifugal Filters. The AMAS/AAV mixture was washed and centrifuged 5 times. The pellet was reconstituted with pH7.4 PBS back to 90 μl. 10 μl KK peptide (20 nM) was added to the reconstituted AMAS/AAV overnight at 4° C. The unreacted KK peptides were removed with Amicon® Ultra Centrifugal Filters. The AMAS/AAV/KK mixture was washed and centrifuged 5 times. The pellet was reconstituted with pH7.4 PBS to 250 μl. 2 μl AMAS peptide (100 nM) was added for 2-hours at room temperature. The unreacted AMAS was removed with Amicon® Ultra Centrifugal Filters. The mixture was washed and centrifuged 5 times. The pellet was reconstituted with pH7.4 PBS to 50 μl. 50 μl PS-SH small molecule (1 μM) was added and the mixture for overnight reaction at 4° C. Unreacted PS-SH small molecules were removed with Amicon® Ultra Centrifugal Filters.

### Example 4. In Vitro and In Vivo Transfection of KKPS-AAV8-CMV-GFP

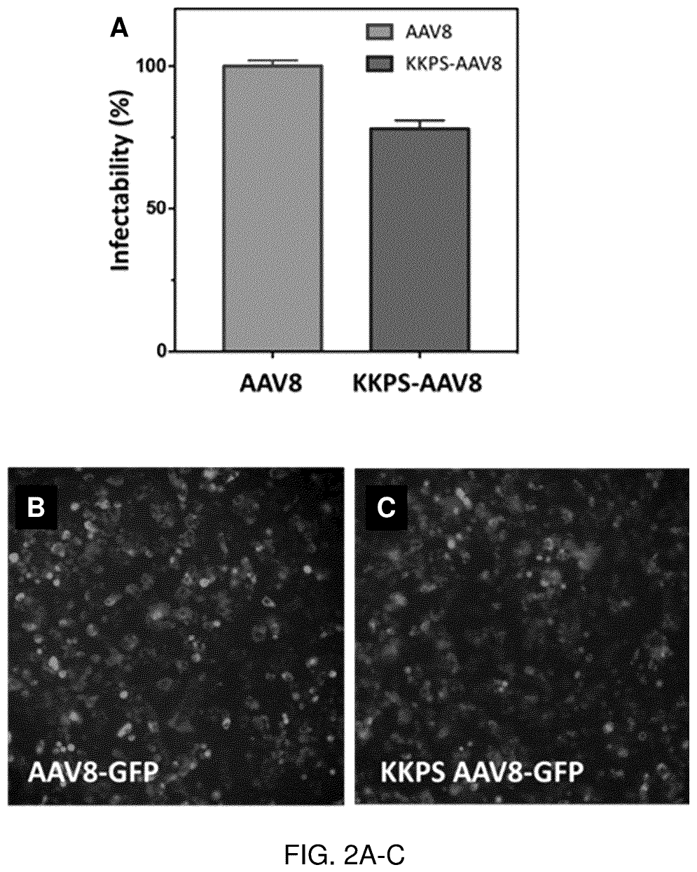

293T cells were cultured in DMEM (Media Tech) supplemented with 10% bovine growth serum, 100 IU/mL penicillin, and 100 mg/ml streptomycin. The cultures were grown in a humidified incubator at 37° C. with 5% CO2. About 10,000 cells per well were plated in 100 μL of media in a 96-well plate. Immediately after plating, the cells were infected with AAV8-GFP or PS modified AAV8-GFP at a multiplicity of infection (MOI) of 50,000 viral genomes per cell. At 24 hours, an additional 100 μL of media was added to cells. At 48 hours post infection, the cells were harvested and analyzed for GFP expression on a FACS Caliber. FIG. 2A shows a graph of percent infectability with the KKPS-AAV8-GFP showing about 75% infectability as compared to the AAV8-GFP control.

In order to show in vivo transfection of the generated KKPS-AAV8-CMV-GFP, male C57B/6 mice were purchased from Jackson laboratory. At 5-6 weeks of age the animals were IV injected with 4×1012 vg/kg of AAV-CMV-GFP or KKPS-AAV-CMV-GFP. After 3 weeks of expression, the mice were sacrificed, and liver sections were sliced and observed by confocal microscopy. FIG. 2B represents the liver section from the AAV-CMV-GFP control group while FIG. 2C represents the KKPS-AAV-CMV-GFP mice. As can be seen in FIG. 2, KKPS-AAV8 kept most of the efficiency of native AAV8.

### Example 5. Brain Targeting Gene Delivery of KKPS-AAV PHP.eb-CAG-eGFP in C57B/6 Mice

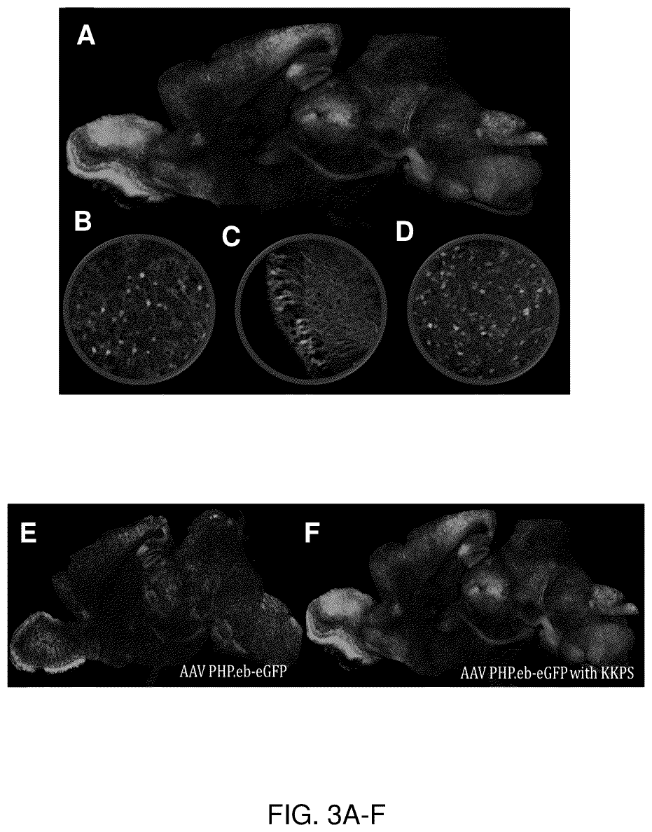

To verify that the generated KKPS-AAV vectors were able to target the brain, and to determine if the KKPS conjugation strategy could furtherly apply to other AAV vectors, we modified a brain targeting AAV vector, php.eb, with the KKPS-peptide. KKPS-AAV PHP.eb-CAG-eGFP vectors and AAV PHP.eb-CAG-eGFP control vectors were administered intravenously to adult male mice (6-8 weeks of age) via retro-orbital injection at doses of 1×1011 vg with 6 weeks of in vivo expression. After 6 weeks of expression, the mice were anesthetized with Euthasol (pentobarbital sodium and phenytoin sodium solution) and transcardially perfused with 30-50 mL of 0.1 M phosphate buffered saline (PBS) (pH 7.4), followed by 30-50 ml of 4% paraformaldehyde (PFA) in 0.1 M PBS. Brains were then harvested and post-fixed in 4% PFA at 4° C. overnight. The tissues were washed and stored at 4° C. in 0.1 M PBS and 0.05% sodium azide using all freshly prepared solutions. 100-μm thick sections of brain were cut on a Leica VT1200 vibratome. GFP was seen throughout the entire brain and in the nerves of the KKPS-AAV.eb-CAG-eGFP tissue samples as shown in FIG. 3A. Representations of the GFP seen throughout cells and neurons in the cortex, hippocampus, and thalamus can be seen in FIG. 3B, FIG. 3C, and FIG. 3D, respectively. Additionally, in comparing whole brain eGFP expression delivered by AAV php.eb-CAG-eGFP vectors (FIG. 3E) and by KKPS-AAV php.eb-CAG-eGFP vectors (FIG. 3F), it appears that the KKPS modified viral vector is at least, if not more, pronounced than what is seen in the AAV-php.eb-CAG-eGFP samples. The GFP seen in the brain tissue is indicative of the ability of the KKP-AAV.eb-CAG-eGFP vector to cross the blood brain barrier while maintaining functional capacity.

### Example 6. Liver Targeting Gene Delivery of KKPS-AAV8-CMV-Fluc in C57B/6 Mice

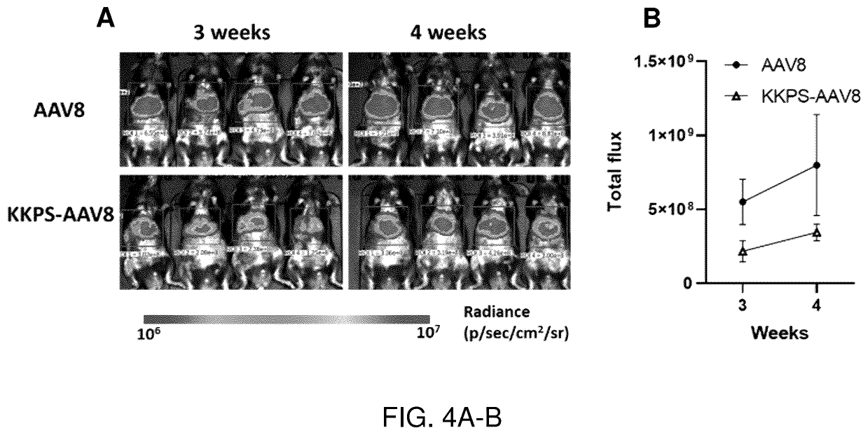

To show the ability of the generated KKPS-AAV vector to target liver, male C57B/6 mice were purchased from the Jackson laboratory. At 5-6 weeks of age the animals were IV injected with 1×1011 particles of AAV-CMV-luciferase or KKPS-AAV-CMV-luciferase. After 3 weeks, the mice were anesthetized with 2-5% isoflurane in oxygen. The d-luciferin substrate was injected intraperitoneally, at a dose of 150 μg/g of body weight. The mice were then placed in a light-tight chamber, and bioluminescence were recorded using the IVIS system (Perkin Elmer). The KKPS-AAV8-CMV-Fluc vector subjects showed similar liver gene targeting to that of the control subjects injected with the AAV-CMV-Fluc control. This similar liver targeting gene delivery of the KKPS-AAV8-CMV-Fluc can be seen in FIG. 4A where the top panel shows the control and the lower panel the experimental vector, both imaged at 3 and 4 weeks post viral vector injection. Additionally, FIG. 4B shows a graph summarizing the data seen in FIG. 4.A, both FIG. 4A and 4B show that the KKPS-AAV8-CMV-Fluc has similar liver targeting to the AAV-CMV-Fluc control.

### Example 7. Immunosuppressive Capacity of KKPS-AAV8 in C57B/6 Mice

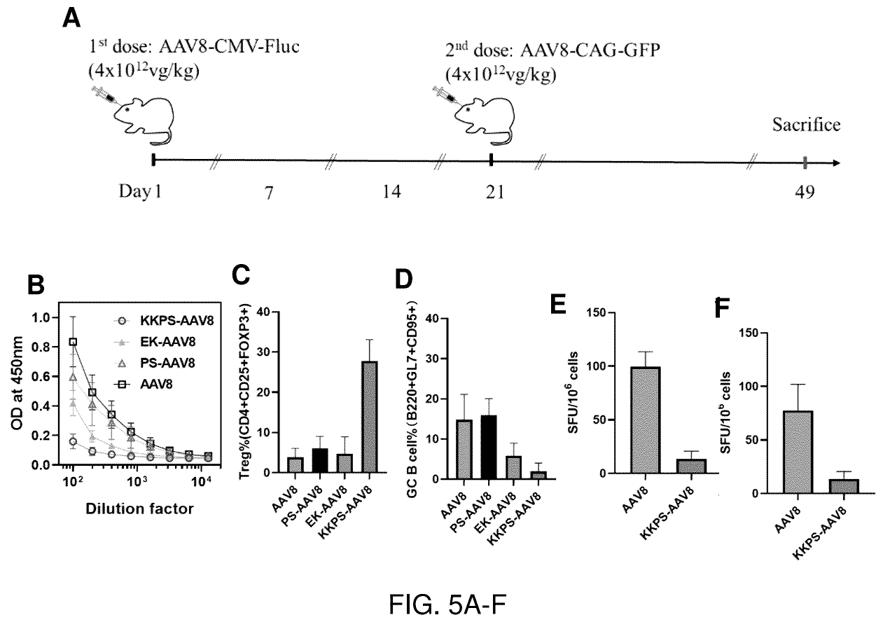

Animals were randomized to treatment groups at the beginning of each study. A sample size of five animals per group was used. C57/BL6 mice (male; body weight, 20 to 30 g) were obtained from the Jackson laboratory. For in vivo immunogenicity study, native AAV8 and KKPS-AAV8 that encoding mCherry (4×1012 vg/kg) were intravenously administered into the mice via retro-orbital injection. After 3 weeks of expression, another dose of native AAV8 and KKPS-AAV8 encoding GFP (4×1012 vg/kg) was IV injected. Starting on day 21 post first intravenously administered vectors, blood was drawn every 7 days until sacrifice. On Day 49, all the mice were euthanized, and their blood collected through cardiac puncture. All blood samples were handled for indirect ELISA test (FIG. 5A). As the first step of direct ELISA test, 1×1010 of AAV8 in 100 μl coating buffer [0.1 M sodium carbonate buffer (pH 10.5)] were used to coat each well of 96-well plates. After overnight coating at 4° C. overnight, the plates were washed five times using PBS-T buffer (pH 7.4) to remove the antigen solutions and then filled with blocking buffer [1% BSA solution in 0.1 M tris buffer (pH 7.4)] for 1-hour incubation at room temperature, subsequent to which the blocking buffer was removed. All wells were then washed by PBS-T buffer five times. Subsequently, serial dilutions of mouse sera in PBS buffer containing 1% BSA were added to the plates (100 μl per well) for 1-hour incubation at 37° C., subsequent to which the mouse sera were removed, and all wells were washed five times with PBS buffer. Next, goat anti-mouse IgM or IgG (HRP-conjugated) secondary antibody was added into each well for another 1-hour incubation at 37° C. After incubation with secondary antibody, all the wells were washed five times using PBS buffer before the addition of 100 μl per well HRP substrate 3,3′,5,5′-tetramethylbenzidine. The plates were shaken for 15 min, and 100 μl of stop solution (0.2 M H2SO4) was added to each well. Absorbance at 450 nm (signal) and 570 nm (background) was recorded by a microplate reader. FIG. 5B shows KKPS successfully inhibited the generation of anti-AAV8 antibody while native AAV8 showed high antibody titers (>6400). Mouse sera naïve to the administration of AAV samples were used as the negative control for all ELISA detections. Mouse spleens were harvested for the isolation of splenocytes by 100 μm cell strainer (Fisherbrand). AAV8-specific mouse Interferon gamma ELISPOT and Anti-AAV8 IgG secreting B cell ELISPOT were performed by using commercially available kits (abcam). The KKPS successfully inhibited the generation and activation of AAV8 specific T cells and B cells as is seen in FIG. 5E. Additionally, part of mouse splenocytes from each group were also cultured in a 12-well plate (106 per well) and restimulated with AAV8 peptide pool. After 72 hours, cells were stained with antibodies for analysis by flow cytometry. KKPS successfully induced the generation and activation of Treg cells (see FIG. 5C), indicating that mice treated with high numbers of PS moieties modified with AAV8 vectors generated immune tolerance to AAV8 gene vector.

### Example 8. In Vivo Gene Delivery of KKPS-AAV8-FVIII in FVIII Deficient Mice (Hemophilia A, HA)

To verify KKPS peptide could enable re-administration of AAV8 vectors in a real animal model, rAAV8-HLP-hFVIII were prepared and loaded in AAV8 vectors and tested the potency in FVIII knockout mice. Animals were randomized to treatment groups at the beginning of each study. A sample size of five animals per group was used. FVIII deficient mice (male; body weight, 20 to 30 g) were obtained from the Jackson laboratory. For in vivo immunogenicity study, native AAV8 and KKPS-AAV8 that encoding luciferase (4×10¹² vg/kg) were intravenously administered into the mice via retro-orbital injection. After 3 weeks of expression, native AAV8 and AAV8 and KKPS-AAV8 encoding hFVIII (4×1012 vg/kg) was IV injected. On Day 49 and 56, plasma samples were collected in sodium citrate treated tubes. Because FVIII serves as a cofactor for the enzyme factor IXa in its activation of the zymogen FX, a commercial FXa chromogenic-based kit (Chromogenix) was used to measure hFVIII activity in sodium citrate-anticoagulated murine plasma samples. The chromogenic FXa assay indirectly measures the total FVIII activity resulting hFVIII produced from AAV8-FVIII vectors.

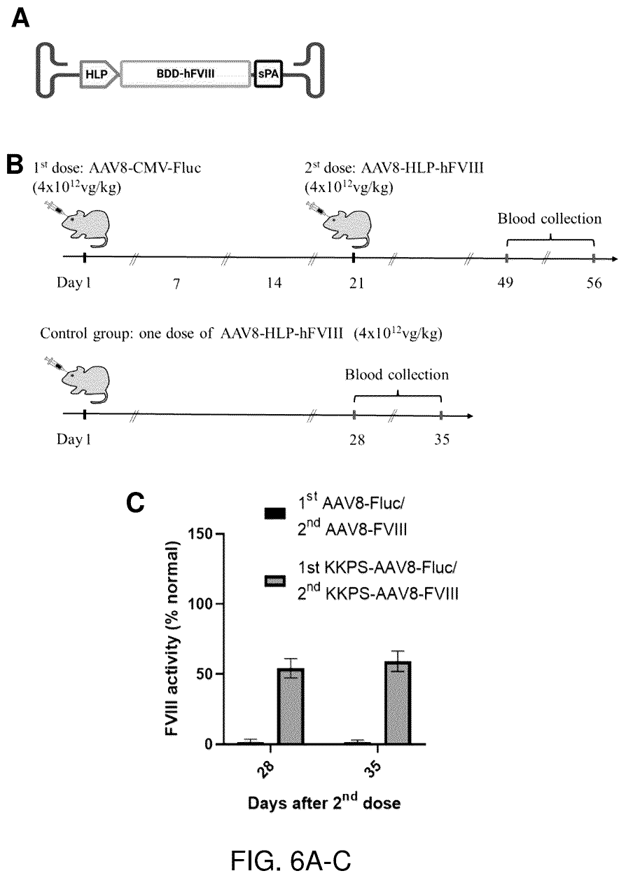

FIG. 6A shows administration route of two-dose cohort in HA mice. FIG. 6B shows FVIII activity in HA mice plasma treated with AAV8-FVIII or KKPS-AAV8-FVIII. Data was normalized with FVIII activity in fresh plasma collected from C57B/6 mice. Due to the immunogenicity of AAV8, second dose of AAV8 could not express FVIII in HA mice. KKPS-AAV8 achieved second dose expression of FVIII in HA mice.

### Example 9. Preparation of PS-Containing Zwitterionic Peptide (PSZP-SH)

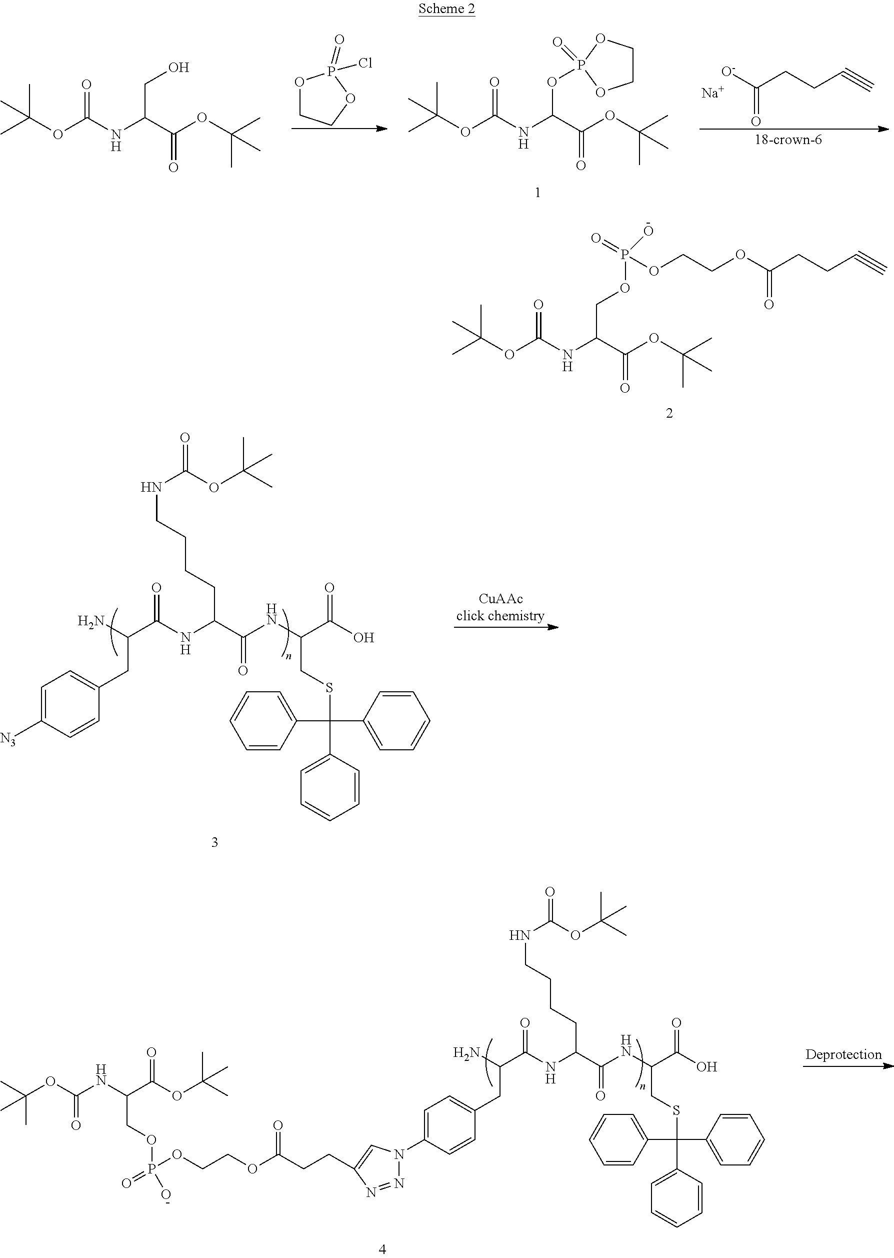

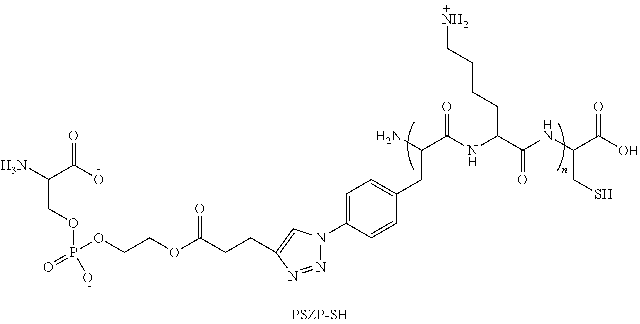

S-alkyne will be prepared as shown in Scheme 2. N-Boc-Ser-OtBu will be dissolved in dry benzene and the solution will be cooled to 0° C. Triethylamine will be then added followed by dropwise addition of 2-chloro-2-oxo-1,3,2-dioxaphospholane and the reaction will be kept stirring at room temperature for 3 hours. After completion of the reaction, diethyl ether will be poured into the reaction mixture and formed salts will be filtered off. The filtrate (containing intermediate 1) will be then concentrated and directly used to react with sodium propiolate (1.5 molar equivalent to starting material) in the presence of 18-crown-6 ether. The product (compound 2) will be purified via flash column chromatography. In the meantime, full protected peptide H-[Phe(azido)-Lys(Boc)]n-Cys(trt)-OH will be synthesized by Fmoc solid-phase peptide synthesis (SPPS) on Liberty Blue automated microwave-assisted peptide synthesizer (CEM). The sequence synthesis scale will be set at 2.5 mmol on trityl chloride resin. Deprotection and coupling reactions will be performed according to default settings provided from CEM. Cleavage will be performed using 20 ml of cocktail (volume mixture of acetic acid/TFE/DCM: 1/1/8) for 30 min at room temperature. Crude full protected peptide (3) will be then precipitated out in the excess cold ether and furtherly purified by preparative-HPLC (Agilent). The PSZP-SH will be obtained through a two-step reaction: 1, copper(I)-catalyzed alkyne-azide cycloaddition (CuAAc) click chemistry; 2, TFA deprotection. The same precipitation in the ether and HPLC purification process will be used to purify the final product. H NMR and LC-MS will be used to confirm the structure and purity of all the compounds and peptides.

### Example 10. Modification of AAV with PSZP-SH

AAVs will be modified with PSZP-SH by a Thiol-ene ‘click’ chemistry. First, surface amine groups of AAV (2E11 vg/ml in pH7.4 PBS with 0.001% Pluronic® F-68) will be converted into maleimide groups by a commercial crosslinker ‘AMAS’ [2 ul AMAS solution (10 nM in DMSO)]. Second, PSZP-SH (10 ul of 20 nM PBS solution) will be conjugated with activated AAVs through the ‘click’ chemistry reaction between the terminal thiol group and the maleimide moiety. Between every reaction step, AAVs will be passed through a desalting column, followed by ultrafiltration to remove unreacted reagents and concentrate the product. Possible loss of infectious titers of AAVs before and after conjugation will be monitored by qPCR. The composition of final products will be characterized by SDS-PAGE and MALDI-TOF.

### Example 11. Preparation of Polysialic Acid Conjugated AAV via Maleimide-Thiol Reaction

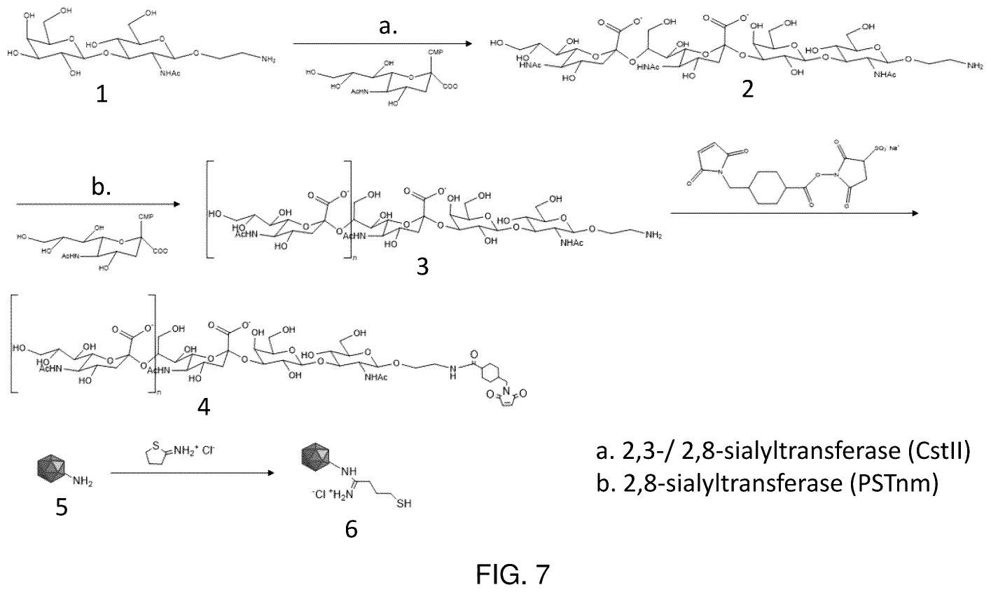

The PSA-NH2 and modified AAV will be prepared as shown in FIG. 7.

The first reaction will utilize the α2,3-/α2,8-sialyltransferase (CstII) to add 1-2 Neu5Ac onto the sugar end of the acceptor 1 2-Aminoethyl 2-acetamido-2-deocy-3-O-β-D-galactopyranosyl-β-D-glucopyranoside, where CMP-Neu5Ac works as a donor substrate (3). The second reaction will allow more Neu5Ac to grow on the sugar end of compound 1 by using α2,8-sialyltransferase (PSTnm) (4). The reason for a two-step enzymatic reaction is that PSTnm requires at least 2 Neu5Ac at the terminal to allow successful polysialylation. The corresponding product 3 will be purified by high performance liquid chromatography and go through a mass spectrometer to quantify the amount of Neu5Ac residues bound. Three chemical reactions can help bind this PSA to the AAVs capsid surface. Sulfo-SMCC will react with compound 3 to modify the amine end to become a maleimide. The amine group on the capsid surface as demonstrated on compound 5 will be turned into thiol group after the reaction with Traut's reagent. The modified 4 and 6 will be conjugated to each other via maleimide-thiol reaction. The conjugation can be analyzed by western blot using a polyclonal antibody against the capsid proteins. The different capsid protein molecular weights between the modified ones versus the native ones can indicate a successful conjugation. AAVs will be purified by ultracentrifuge and gone through a desalting gravity column to clear out the reactants and salts.

### Example 12. Preparation of Polysialic Acid Conjugated AAV via Click Reaction

The starting acceptor compound will be a similar disaccharide with an azide group due to the specificity of the enzymes. PSA will be produced by the same enzymatic reactions. The AAVs will be modified by 2,5-dioxopyyrrolidin-1-yl pent-4-ynoate to obtain an alkyne group. The AAVs and the PSA will be conjugated to each other by click chemistry under the catalyzation by CuSO4, THPTA, and sodium I-ascorbate. However, the starting compound can also be commercially bought alkyne-linked glycans with Neu5Ac at the sugar terminal. In this case, only the PSTnm enzymatic reaction will be required to grow PSA onto the glycan. Similar conjugation method and characterization method will apply. HPLC will be used to purify the final PSA compound, and H NMR and LC-MS will be used to assess the structure and molecular weight of the compound. Centrifugation and size exclusion column will be used to purify AAVs. The conjugation after click chemistry will be characterized by SDS-PAGE.

What is claimed is:

1. A modified viral vector, comprising:
a viral vector (VV); and
an immunosuppressive moiety (ISM) covalently linked directly or through a linker to the viral vector.

2. The modified viral vector of claim 1, wherein the viral vector is a virus selected from the group consisting of retroviruses, lentiviruses, adenoviruses, and adeno-associated viruses (AAV).

3. The modified viral vector of any one of the preceding claims, wherein the viral vector is an adeno-associated virus (AAV) selected from the group consisting of AAV1, AAV2, AAV3, AAV4, AAV5, AAV6, AAV7, AAV8, AAV9, AAV10, AAV11, AAV6.2, AAVrh10, AAV-DJ, AAV-DJ/8, AAV-PHP.B, AAV-PHP.eB, AAV-PHP.S, AAV2-retro, AAV2-QuadYF, AAV2.7m8, and genetically engineered derivatives thereof.

4. The modified viral vector of any one of the preceding claims, wherein the immunosuppressive moiety (ISM) comprises one or more compounds selected from the group consisting of small molecules, polymeric molecules, and peptides, wherein the small molecules, polymeric molecules, and peptides have a molecular weight of 100-10,000 g/mol.

5. The modified viral vector of any one of claims 1-4, wherein the immunosuppressive moiety (ISM) comprises a phosphoserine (PS) having the following structure: 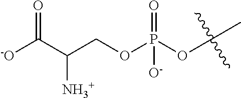
wherein the wavy line indicates a bond to a linker or a direct bond to the viral vector.

6. The modified viral vector of any one of claims 1-4, wherein the immunosuppressive moiety (ISM) comprises polysialic acid (PSA).

7. The modified viral vector of claim 6, wherein the PSA comprises the following structure: 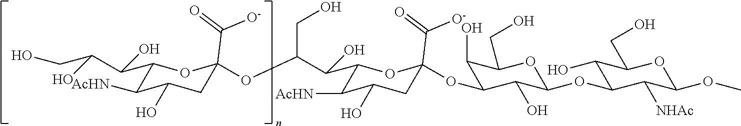
wherein Ac represents acetyl; and
n is at least 2.

8. The modified viral vector of any one of the preceding claims, wherein the immunosuppressive moiety (ISM) comprises one or more mTOR inhibitors.

9. The modified viral vector of claim 8, wherein the one or more mTOR inhibitor is selected from the group consisting of Rapamycin, Temsirolimus, Everolimus, Umirolimus, and combinations thereof.

10. The modified viral vector of any one of the preceding claims, wherein the immunosuppressive moiety comprises one or more selected from the group consisting of, aryl hydrocarbon receptor (AHR) ligands, vitamin D3, retinoic acid, peptides with CxxC/CxxS flanking epitope where x is any amino acid, and combinations thereof.

11. The modified viral vector of any one of the preceding claims, wherein the immunosuppressive moiety comprises one or more molecules from apoptotic cells.

12. The modified viral vector of claim 11, wherein the one or more molecules from apoptotic cells are selected from the group consisting of phosphatidylserine, chromatin oligonucleotide, and combinations thereof.

13. The modified viral vector of any one of the preceding claims, wherein the immunosuppressive moiety comprises one or more secondary lymphoid organs (spleen or lymph nodes) or liver targeting moieties.

14. The modified viral vector of claim 13, wherein the one or more secondary lymphoid organ or liver targeting moieties are selected from the group consisting of: N-acetylgalactosamine (GalNAc), N-Acetylglucosamine (GlcNAc), N-acetylneuraminic acid (NeuAc or sialic acid), galactose, and fucose, and combinations thereof.

15. The modified viral vector of any one of the preceding claims, wherein the immunosuppressive moiety comprises one or more inflammation reducing moieties.

16. The modified viral vector of claim 15, wherein the inflammation reducing moieties are selected from Z2-Y12, Z1-Y15, Z1-Y19, dexamethasone, lymphocyte function-associated antigen antagonist, d-mannose, and combinations thereof.

17. The modified viral vector according to any one of claims 1-16, wherein the viral vector and immunosuppressive moiety are covalently linked to each other directly.

18. The modified viral vector according to any one of claims 1-16, wherein the viral vector and immunosuppressive moiety are covalently linked through a linker.

19. The modified viral vector of claim 18, wherein the linker comprises a linker peptide and/or a crosslinker compound.

20. The modified viral vector of claim 19, wherein the linker peptide is a peptide of 25 amino acids or less.

21. The modified viral vector of claim 19 or 20, wherein the linker peptide comprises alternating Glu-Lys (EK) peptides or Lys-Lys (KK) peptides.

22. The modified viral vector of claim 19 or 20, wherein the linker peptide comprises (KK)8-C-NH2, or a derivative thereof.

23. The modified viral vector of any one of claims 19-22, wherein the crosslinker compound comprises a N-hydroxysuccinimide ester-Maleimide heterobifunctional aliphatic reagent.

24. The modified viral vector of any one of claims 19-23, wherein the crosslinker compound is selected from the group consisting of AMAS, BMPS, GMBS, Sulfo-GMBS, MBS, Sulfo-MBS, SMCC, Sulfo-SMCC, EMCS, Sulfo-EMCS, SMPB, Sulfo-SMPB, SMPH, LC-SMCC, and Sulfo-KMUS.

25. The modified viral vector according to any one of claims 19-24, wherein the modified viral vector comprises a plurality of the linker.

26. The modified viral vector according to any one of claims 19-25, wherein each linker comprises a plurality of a peptide linker and/or a plurality of a crosslinker compound.

27. The modified viral vector of any one of claims 19-26, wherein the viral vector comprises surface sites to which the immunosuppressive moiety or the linker covalently binds.

28. The modified viral vector of claim 27, wherein the surface sites of the viral vector comprise capsid proteins, gag proteins, envelop proteins, and/or lipid layers.

29. The modified viral vector of any one of claims 1-16, wherein the modified viral vector comprises the following structure: 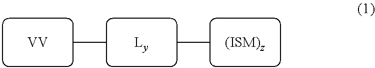
wherein:
VV is the viral vector;
L is a linear or branched linker selected from the group consisting of peptides, saccharides, lipids, and non-biological molecules and polymers, wherein y is 0 or 1, which corresponds to the absence or presence of the linker, respectively;
ISM is the immunosuppressive moiety; and
z is at least 1, wherein z corresponds to the number of ISM attached to L;
wherein the lines connecting VV, L_{y}, and ISM represent covalent bonds.

30. The modified viral vector of any one of claims 1-16 and 29, wherein the linker or ISM is attached to the viral vector via an amino group of the viral vector.

31. The modified viral vector of claim 30, wherein the amino group is on a capsid or envelope of the viral vector.

32. The modified viral vector of any one of claims 1-16 and 29-31, wherein the linker is present and the modified viral vector comprises the following structure:

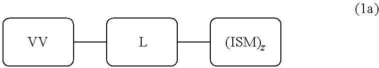

33. The modified viral vector of claim 32, wherein the linker is attached to the viral vector via an amino group of the viral vector.

34. The modified viral vector of claim 33, wherein the amino group is on a capsid or envelope of the viral vector.

35. The modified viral vector of any one of claims 1-16 and 29-34, wherein the modified viral vector comprises the following structure: 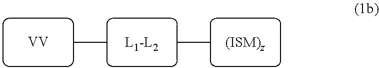
wherein:
VV is the viral vector;
L₁ and L₂ are portions of the linear or branched linker L, wherein L₁ represents a bifunctional crosslinker possessing an amino-reactive and thiol-reactive group, wherein the amino reactive group is bound to an amino group of the viral vector; and L₂ represents a linking portion containing a thiol group bound to the thiol-reactive group of L₁, wherein L₂ is also bound to the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

36. The modified viral vector of claims 1-16 and 29-35, wherein the modified viral vector comprises the following structure: 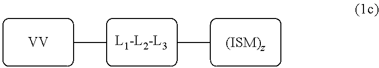
wherein:
VV is the viral vector;
L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is selected from the group consisting of peptides, saccharides, lipids, and non-biological molecules and polymers; and L₃ represents a bifunctional crosslinker possessing an amino-reactive and thiol-reactive group, wherein the amino reactive group is bound to an amino group of L₂ and the thiol-reactive group is bound to a thiol group of the ISM, or wherein the amino reactive group is bound to an amino group of the ISM and the thiol-reactive group is bound to a thiol group of L₂;
ISM is the immunosuppressive moiety; and
z is at least 1, wherein z corresponds to the number of ISM attached to L.

37. The modified viral vector of any one of claims 1-16 and 29, wherein the modified viral vector comprises the following structure: 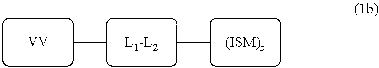
wherein:
VV is the viral vector;
L₁ and L₂ are portions of the linear or branched linker L, wherein VV is modified to contain a thiol group, and L₁ represents a thiol-reactive group bound to the thiol group of VV, wherein L₂ is bound to L₁ and the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

38. The modified viral vector of any one of claims 1-16 and 29, wherein the modified viral vector comprises the following structure: 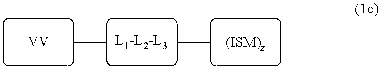
wherein:
VV is the viral vector;
L₁, L₂, and L₃ are portions of the linear or branched linker L, wherein VV is modified to contain a thiol group, and L₁ represents a thiol-reactive group bound to the thiol group of VV; L₃ represents a bifunctional crosslinker possessing an amino-reactive and thiol-reactive group, wherein the amino reactive group is bound to an amino group of L₂ and the thiol-reactive group is bound to a thiol group of the ISM, or the amino reactive group is bound to an amino group of the ISM and the thiol-reactive group is bound to a thiol group of L₂; wherein L₂ is selected from the group consisting of peptides, saccharides, lipids, and non-biological molecules and polymers;
ISM is the immunosuppressive moiety; and
z is at least 1, wherein z corresponds to the number of ISM attached to L.

39. The modified viral vector of any one of claims 1-16 and 29, wherein the modified viral vector comprises the following structure: 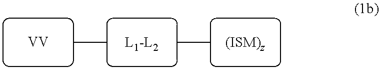
wherein:
VV is the viral vector;
L₁ and L₂ are portions of the linear or branched linker L, wherein VV and L₂ are modified to contain an azide or alkyne group in order for VV and L₂ to attach by azide-alkyne cycloaddition click chemistry;
L₁ represents a 1,2,3-triazole group connecting VV and L₂, wherein the 1,2,3-triazole group is the result of an azide-alkyne cycloaddition click chemistry reaction between the azide or alkyne group on the VV and alkyne or azide group, respectively, on L₂; wherein L₂ is bound to L₁ and the ISM, and L₂ is selected from the group consisting of peptides, saccharides, lipids, and non-biological molecules and polymers;
ISM is the immunosuppressive moiety; and
z is at least 1, wherein z corresponds to the number of ISM attached to L.

40. The modified viral vector of any one of claims 1-16 and 29, wherein the modified viral vector comprises the following structure: 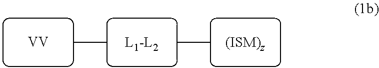
wherein:
VV is the viral vector;
L₁ and L₂ are portions of the linear or branched linker L, wherein L₁ and ISM are modified to contain an azide or alkyne group in order for L₁ and ISM to attach by azide-alkyne cycloaddition click chemistry;
L₁ is selected from the group consisting of peptides, saccharides, lipids, and non-biological molecules and polymers; L₂ represents a 1,2,3-triazole group connecting L₁ and ISM, wherein the 1,2,3-triazole group is the result of an azide-alkyne cycloaddition click chemistry reaction between the azide or alkyne group on L₁ and alkyne or azide group, respectively, on ISM;
ISM is the immunosuppressive moiety; and
z is at least 1, wherein z corresponds to the number of ISM attached to L.

41. The modified viral vector of any one of claims 29-40, wherein the ISM comprises a phosphoserine (PS) having the following structure: 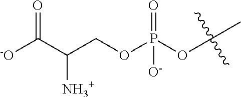
wherein the wavy line indicates a bond to a linker or a direct bond to the viral vector.

42. The modified viral vector of claims 1-16 and 29-35, wherein the modified viral vector comprises the following structure: 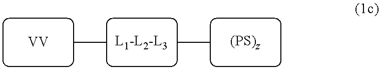
wherein:
VV is the viral vector;
phosphoserine (PS) is modified to contain a thiol group, and multiple PS moieties are present;
z is greater than 1 and corresponds to the number of PS moieties attached to L₂ via L₃;
L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is a polypeptide containing multiple amino groups; and L₃ represents a multiplicity of bifunctional crosslinkers each possessing an amino-reactive and thiol-reactive group, wherein the amino reactive group is bound to the amino groups of L₂ and the thiol-reactive group is bound to the thiol groups of the multiple PS moieties.

43. The modified viral vector of claims 1-16 and 29-35, wherein the modified viral vector comprises the following structure: 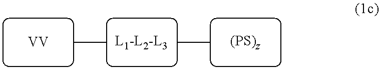
wherein:
VV is the viral vector;
phosphoserine (PS) is modified to contain a thiol group;
L₁, L₂, and L₃ are portions of the linear or branched linker L, wherein L₁ represents a bifunctional crosslinker possessing an amino-reactive and thiol-reactive group, wherein the amino reactive group is bound to an amino group of the viral vector; L₂ represents a linking portion containing a thiol group bound to the thiol-reactive group of L₁, and L₂ is a polypeptide; L₂ and PS are modified to contain an azide or alkyne group in order for L₂ and PS to attach by azide-alkyne cycloaddition click chemistry and L₃ represents a 1,2,3-triazole group connecting L₂ and PS, wherein the 1,2,3-triazole group is the result of an azide-alkyne cycloaddition click chemistry reaction between the azide or alkyne group on L₂ and alkyne or azide group, respectively, on PS; and
z is at least 1, wherein z corresponds to the number of PS attached to L₂.

44. The modified viral vector of any one of claims 29-40, wherein the immunosuppressive moiety (ISM) comprises polysialic acid (PSA).

45. The modified viral vector of claim , wherein the PSA comprises the following structure: 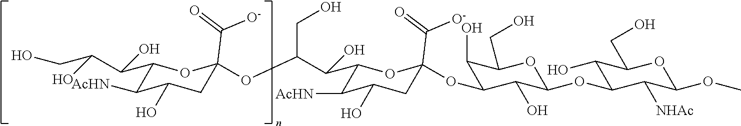
wherein Ac represents acetyl; and
n is at least 2.

46. The modified viral vector of claims 1-16 and 29-35, wherein the modified viral vector comprises the following structure: 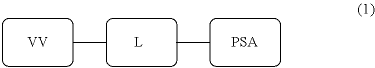
wherein:
VV is the viral vector, modified to contain a thiol group;
PSA is polysialic acid;
L is a linker connecting VV and PSA and comprises a thiol-reactive group bound to the thiol group of VV.

47. The modified viral vector of claims 1-16 and 29-35, wherein the modified viral vector comprises the following structure: 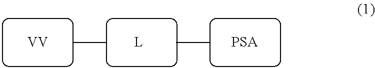
wherein:
VV is the viral vector, modified to contain an alkyne or azide group;
PSA is polysialic acid, modified to contain an alkyne or azide group;
L is a 1,2,3-triazole group connecting VV and PSA, wherein the 1,2,3-triazole group is the result of an azide-alkyne cycloaddition click chemistry reaction between the azide or alkyne group on VV and alkyne or azide group, respectively, on PSA.

48. The modified viral vector of any one of the preceding claims, wherein the linker comprises a peptide.

49. The modified viral vector of claim 48, wherein the peptide comprises polylysine.

50. The modified viral vector of any one of the preceding claims, wherein the peptide contains no more than 25 amino acid units.

51. The modified viral vector of any one of the preceding claims, wherein the modified viral vector comprises more than one immunosuppressive moiety covalently bound to the viral vector.

52. The modified viral vector of any one of the preceding claims, wherein the modified viral vector comprises 1-10,000 immunosuppressive moieties covalently bound to the viral vector.

53. The modified viral vector of any one of the preceding claims, wherein the modified viral vector comprises 1-5,000 immunosuppressive moieties covalently bound to the viral vector.

54. The modified viral vector of any one of the preceding claims, wherein the modified viral vector comprises 1-2,000 immunosuppressive moieties covalently bound to the viral vector.

55. The modified viral vector of any one of the preceding claims, wherein the modified viral vector comprises 100-2,000 immunosuppressive moieties covalently bound to the viral vector.

56. The modified viral vector of any one of the preceding claims, wherein the modified viral vector achieves a transfection efficiency that is at least 30% of the transfection efficiency by an unmodified viral vector.

57. The modified viral vector of any one of the preceding claims, wherein the modified viral vector achieves a transfection efficiency that is at least 40% of the transfection efficiency by an unmodified viral vector.

58. The modified viral vector of any one of the preceding claims, wherein the modified viral vector achieves a transfection efficiency that is at least 50% of the transfection efficiency by an unmodified viral vector.

59. The modified viral vector of any one of the preceding claims, wherein the modified viral vector achieves a transfection efficiency that is at least 60% of the transfection efficiency by an unmodified viral vector.

60. The modified viral vector of any one of the preceding claims, wherein the modified viral vector achieves a transfection efficiency that is at least 70% of the transfection efficiency by an unmodified viral vector.

61. A method for preparing a modified viral vector, the method comprising attaching an immunosuppressive moiety to a viral vector to obtain a modified viral vector according to any of the preceding claims.

62. A method for introducing genetic material into a cell, the method comprising contacting a cell with at least one modified viral vector according to any one of claims 1-60.

63. The method of claim 62, wherein the cell is contacted multiple times with the at least one modified viral vector.

64. The method of any one of claims 62-63, wherein the at least one modified viral vector comprises multiple modified viral vectors.

65. A method for treating a subject, the method comprising administering to a subject at least one modified viral vector according to any one of claims 1-60.

66. The method of claim 65, wherein the subject exhibits a reduced immune response after the subject is administered the at least one modified viral vector as compared to a control subject administered an unmodified viral vector.

67. The method of any one of claims 65-66, wherein the at least one modified viral vector is administered multiple times to the subject.

68. The method of any one of claims 65-67, wherein the at least one modified viral vector comprises multiple modified viral vectors.

69. The method of claim 67 or 68, wherein the subject exhibits a reduced immune response as compared to a control subject administered an unmodified viral vector.

70. The method of claim 67 or 68, wherein the subject is administered with (i) a modified viral vector of any one of claims 1-60 at a first point in time and subsequently (ii) the modified viral vector at a second point in time, and the subject exhibits a reduced immune response as compared to a control subject administered an unmodified viral vector at the first and second points in time.

71. The method of any one of claims 66-67, wherein the subject is administered with (i) a modified viral vector of any one of claims 1-60 at a first point in time, and subsequently (ii) a different modified viral vector of any one of claims 1-60 at a second point in time; and the subject exhibits a reduced immune response as compared to a control subject administered an unmodified viral vector at the first and second points in time.

72. The method of claim 70 or 71, wherein the second point in time is between 1 day and 49 days after the first point in time.

73. The method of any one of claims 70-72, wherein the second point in time is at least 21 days after the first point in time.
